(19) United States
(12) Patent Application Publication
(10) Pub. No.: US 2009/0186838 A1
(43) Pub. Date: Jul. 23, 2009

(54) Amphotericin Derivatives

(75) Inventors: Erick Carreira, Zumikon (CH); Valerie Paquet, Montreal (CA)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Eidgenössische Technische Hochschule Zürich, Zürich (CH)

(21) Appl. No.: 12/224,355

(22) PCT Filed: Feb. 21, 2007
(86) PCT No.: PCT/EP2007/001468

§ 371 Date: Nov. 18, 2008

(30) Foreign Application Priority Data
Feb. 23, 2006 (EP) 06003654.8

(51) Int. Cl. A61K 31/7048; C07H 17/08; A61P 31/10

(52) U.S. Cl. 514/31; 536/6.5

(57) ABSTRACT

The present invention provides new polyene macrolide derivatives which show very low toxicity while retaining high antifungal activity as compared with amphotericin B (AmB). These polyene macrolide derivatives comprise a polyene macrolide backbone having at least one free amino group, wherein the amino group is doubly alkylated with at least one hydrocarbon group carrying a total of at least two basic groups.

### FIELD OF THE INVENTION

The present invention relates to new polyene macrolide derivatives and salts thereof, their pharmaceutical compositions optionally in combination with other active agents, methods of making these new polyene macrolide derivatives as well as their uses in the treatment and prevention of fungal infections.

### BACKGROUND OF THE INVENTION

Fungal infections represent a serious problem in particular for patients with immune systems compromised either by HIV infection, or administration of immunosuppressive drugs during cancer therapy and organ transplantation. Due to high dissemination and proliferation rates of many pathogenic fungi along with their insusceptibility to common antimicrobial drugs there exists an urgent need for efficient and reliable antifungal therapy. Up to date, polyene macrolide antibiotics proved to be the most effective antifungal agents due to their potent fungicidal activity, broad spectrum, and relatively low frequency of resistance among the fungal pathogens. It has been shown that the polyene macrolide antibiotics have selectivity for inhibiting organisms whose membranes contain sterols. Their mechanism of action is, at least in part, dependent upon their binding to a sterol moiety, primarily ergosterol, present in the membrane of sensitive fungi. Once this interaction occurs, the polyenes appear to form pores or channels in the fungal cell membrane which results in an increase of permeability of the membrane and the leakage of a variety of small molecules such as potassium and other ion and solute components out of the cell. This disruption in membrane integrity ultimately leads to cell death. Typical uses for conventional polyenes such as amphotericin B (AmB, FIG. 1) and nystatin include treatment of fungal diseases such as those resulting from Candida infections (e.g., C. albicans, C. tropicalis, etc.) as well as a variety of other diseases such as histoplasmosis, coccidioidomycosis, systemic sporotrichosis, aspergillosis, mucormycosis, chromablastomycosis, blastomycosis and cryptococcosis. Yet, polyene macrolides are also known for their high insolubility and high toxicity leading to serious side effects including renal failure, hypokalemia and thrombophlebitis.

Attempts to minimize these serious side effects and to overcome the low solubility of polyene-macrolides have led to the development of new polyene macrolide derivatives with desired improved characteristics as well as suitable pharmaceutical formulations, in particular lipid-based formulations. Apart form the conventional oral (Fungilin®) and intravenous (Fungizone®, a colloidal suspension) AmB formulations, three lipid based formulations have currently been developed to ameliorate the serious side effects. These include a colloidal dispersion (Amphocil® or Amphotec®), a lipid complex (Abelect®) and a liposomal complex (Ambisome®, Fungisome®), all of which have been reported to show a similar spectrum of activity but reduced toxicities (J. Brajtburg, J. Bolard, Clin. Microbiol. Rev. 1996, 9, 512-531; Lemke A. et al, Appl. Microbiol. Biotechnol. 2005, 68, 151-162; J. P. Adler-Moore, R. T. Proffitt, J. Liposome Res. 1993, 393, 429-450). Yet, a major disadvantage is that such lipid-based formulations are very costly (up to 50 times more than AmB).

To address these limitations there have been many attempts to generate more active polyene macrolide derivatives through simple structural modifications, in particular through functionalizations at the amine in the mycosamine appendage of AmB (FIG. 1) and/or at the C-16 carboxylate.

Modifications at the amino group include for example N-glycosylation with mono-, oligosaccharides (U.S. Pat. No. 4,093,796, Falkowski et al.), N-glycosylation with 1-amino-1-deoxyketose and derivatives thereof (U.S. Pat. No. 5,314,999), N-methylglucamine salt formation of N-glycosyl derivatives (U.S. Pat. No. 4,294,958, Falkowski et al.), formation of mixed N-alkyl-N-glycosyl derivatives (U.S. Pat. No. 5,942,495), N-trimethylammonium salt formation (U.S. Pat. No. 4,294,958, Falkowski et al), formation of alkyl- or arylamides (U.S. Pat. No. 4,783,527, Falkowski et al., U.S. Pat. No. 5,298,495), N-substitution with aminoacyl-groups (U.S. Pat. No. 4,272,525), and, formation of N-guanidine derivatives (U.S. Pat. No. 4,396,610).

Modifications at the C16-group optionally in combination with N-derivatization include for example formation of N-glycosylated C-16 carboxylester, -thioester or -amides (WO 01/51061; WO 01/91758), formation of N-acetyl or N-alkylene substituted C-16 alkylesters (U.S. Pat. No. 4,365,058, Falkowski et al), derivatization at the C16-position with concomitant amino group protection (WO 93/17034, WO 93/16090, WO 93/14100 and references sited therein), derivatization of the C-16 position with poly(ethylene glycols) (WO 96/32404), C-16 dimerization (Matsumori, N. et al., J. Am. Chem. Soc. 2002, 124, 4180-4181; Yamaji, N. et al., Org. Lett. 2002, 4, 2087-2089) and in more recent developments functionalized carbon nanotube derivatives (Wu, W. et al., Angew. Chem. Int. Ed. 2005, 44, 6358-6362).

However, none of the foregoing derivatives could satisfactorily overcome all of the disadvantages described hereinabove. Most modifications of the amine group yielded derivatives displaying diminished activity, because the NH₂ group in the native AmB (FIG. 1) seemed critical and tolerated little alteration.

Similarly, it has been observed that while C-16 ester and amide derivatives generally display reduced hemotoxicity, this usually occurred only at the expense of antifungal activity (Carmody, M. et al., J. Biol. Chem. 2005, 280, 34420-34426; Keim, G. R. et al., Science 1973, 179, 584-585).

Consequently, AmB (FIG. 1) remained despite its manifold drawbacks 40 years after its discovery (U.S. Pat. No. 2,908,611) the therapeutic agent of choice against most systemic mycoses, such as invasive aspergillosis, candidemia (in particular fluconazole-resistant Candida), mucormycosis, fusariosis, and cryptococcosis meningitis.

Thus there is still a great need for the development of improved polyene macrolide derivatives that overcome one or more of the disadvantages associated with the known polyene macrolides and their formulations. These new polyene macrolide derivatives should exhibit high antifungal activities in combination with low toxicity and preferably high water solubility to give pharmaceutical formulations having a sufficiently broad spectrum and/or high degree of efficacy of antifungal activity suitable for safe treatment of fungal diseases. These properties would render them in particular desirable for use in treatment of immunocompromised individuals.

Applicants have now surprisingly found that specific modifications at the primary amino group of the deoxysugar of polyene macrolides, such as the mycosamine moiety of AmB, can lead to potent derivatives, which have shown to be more active for example against a Saccharomyces cerevisiae wild type (wt) strain and especially against an AmB-resistant Candida albicans strain. Moreover, these novel compounds of the invention exhibit significantly lower hemotoxicity when compared to AmB (FIG. 1) and superior selectivity towards ergosterol containing vesicles.

### SUMMARY OF THE INVENTION

The present invention provides new polyene macrolide derivatives that provide significant advantages over the currently used polyene macrolides. Thus in a first aspect, the present invention provides polyene macrolide derivatives that show very low toxicity while retaining high antifungal activity as compared with AmB. The polyene macrolide derivatives of the invention are characterized by a polyene macrolide backbone having at least one free amino group, wherein the amino group is doubly alkylated with at least one hydrocarbon group carrying a total of at least two basic groups. In a specific embodiment a carboxyl-substituent on the macrolide ring (e.g. the C-16 carboxyl-group in AmB) is optionally esterified or amidated.

More specifically the present invention relates to polyene macrolide derivatives according to formula I:

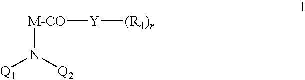

or a pharmaceutically acceptable salt thereof,
wherein:
M represents a polyene macrolide backbone;
Q₁ and Q₂ represent
   (i) a group of formula —(R₁)—(X₁)ₘ and —(R₂)—(X₂)ₙ, respectively, wherein
   X₁, X₂ represent independently of each other a basic group, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
   m, n represent independently of each other 0, 1 or 2, with m+n≧2
   R₁, R₂ represent independently of each other an unsubstituted or substituted hydrocarbon group, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; or
   (ii) taken together with the adjacent nitrogen atom to which they are attached, a nitrogen-containing heterocyclic group substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

In a specific embodiment the polyene macrolide backbone of the polyene macrolide derivatives of the invention may be derived form any known, or not yet known, but later discovered, polyene macrolide backbone having at least one free amino group, such as an amino group of a deoxysugar, including amphotericin B, nystatin, candidin, candicidin, aureofacin, levorin, mycoheptin, partricin, perimycin, pimaricin, polyfungin, rimocidin or trichomycin, preferably amphotericin B, nystatin or pimaricin.

Thus in a specific embodiment the invention is directed to polyene macrolide derivatives according to formula II:

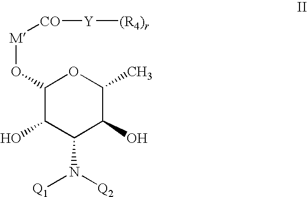

or a pharmaceutically acceptable salt thereof,
wherein:
M′ represents the macrocyclic lactone ring of a polyene macrolide backbone;
and Q₁, Q₂, Y, R₄, and r are as defined hereinabove.

In a further specific embodiment the invention is directed to polyene macrolide derivatives according to formulae III a-c:

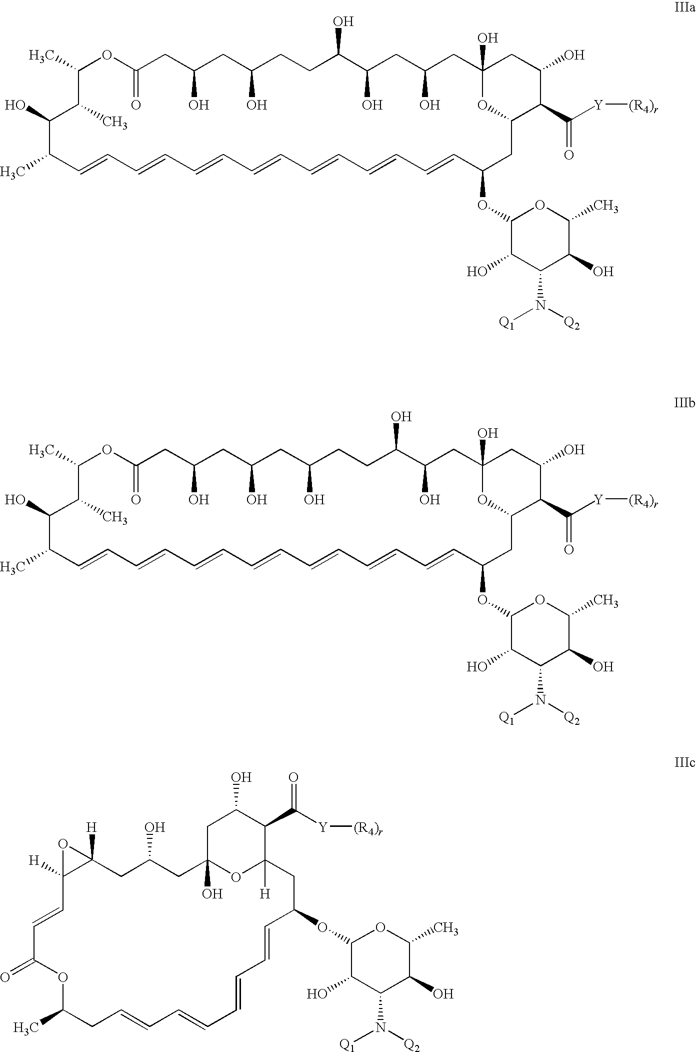

wherein:
Q₁, Q₂, Y, R₄, and r are as defined hereinabove.

In a further aspect, the present invention is directed to a specific subgroup of polyene macrolide derivatives according to formula IV:

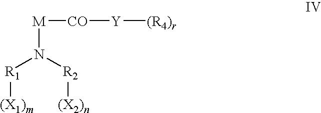

or a pharmaceutically acceptable salt thereof,
wherein:
M represents a polyene macrolide backbone;
R₁, R₂ represent independently of each other linear or branched —(CH₂)ₚ—, wherein p is an integer from 0 to 12 and in which one or more —CH₂— groups are optionally replaced by —O—, —CO—, —COO—, —CONR₅—, —NR₅—, —CH═CH—, wherein R₅ independently represents hydrogen or alkyl;
X₁, X₂ represent independently of each other a basic group, which may be attached to any —CH₂— group of R₁ and R₂, respectively, and is preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

In a further aspect, the present invention is directed to a further subgroup of the polyene macrolide derivatives of the present invention according to formula VII:

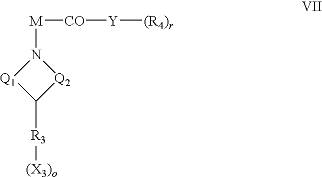

or a pharmaceutically acceptable salt thereof,
wherein:
M represents a polyene macrolide backbone;
Q₁, Q₂ form together with the adjacent nitrogen atom to which they are attached a nitrogen-containing heterocyclic group;
X₃ represents a basic group, which may be attached to any —CH₂-group of R₃, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

In a further specific embodiment the present invention relates to polyene macrolide derivative of formula X:

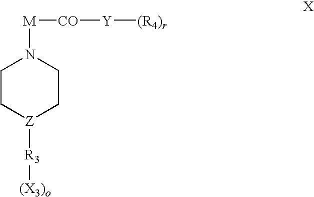

or a pharmaceutically acceptable salt thereof,
wherein:
M represents a polyene macrolide backbone;
Z represents —CH— or —N—;
X₃ represents a basic group, which may be attached to any —CH₂— group of R₃, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2

In a further aspect the invention relates to a method of producing a polyene macrolide derivative according to the invention, comprising subjecting a polyene macrolide to double reductive alkylation using standard chemistry protocols. More specifically, a polyene macrolide may be subjected to double reductive alkylation with two optionally protected functionalized aldehydes P—(X₁)ₘ—(R₁)—CHO and P—(X₂)ₙ—(R₂)—CHO, wherein X₁, X₂, R₁ and R₂ are as defined hereinabove and P is H or a suitable protecting group, to give a compound of the invention wherein the amino group is alkylated with two open chains. Alternatively a polyene macrolide may be subjected to double reductive alkylation with one optionally protected functionalized aldehyde P—(X₃)ₒ—(R₃)—(CHO)₂, wherein X₃ and R₃ are as defined hereinabove and P is H or a suitable protecting group, to give a compound of the invention wherein the amino group is embedded in a ring structure.

The polyene macrolide derivatives of the present invention have significant advantages over currently available polyene macrolide antifungals. Specifically, the polyene macrolide derivatives of the present invention show excellent therapeutic potency against e.g. Saccaromyces cerevisiae as well as AmB resistant strains, e.g. Candida albicans. Furthermore they show both efficient ion channel formation and good selectivity as demonstrated in K+ efflux measurements.

Thus in yet another aspect the present invention provides methods of inhibiting the growth of fungi, such as Candida species (e.g. C. albicans, C. glabrata), Saccharomyces cerevisiae, Aspergillus species, Crytococcusneoformans, Blastomyces dennatitidis, Histoplasmacapsulatum, Torulopsis glabrata, Coccidioidesimmitus, Paracoccidioides braziliensis, and the like, which methods comprise contacting a fungus with an effective amount of a polyene macrolide derivative of the invention, or a pharmaceutically acceptable salt thereof, or a pharmaceutical composition thereof to inhibit the growth of the fungus.

In addition, the polyene macrolide derivatives of the present invention show higher water solubility as compared to AmB, and significantly reduced hemotoxicity.

Thus the present invention provides in a further aspect polyene macrolide derivatives according to the invention (including pharmaceutically acceptable salts and/or pharmaceutical compositions thereof) for use in therapy, in particular for use in the treatment and/or prevention of fungal infections.

Other uses include further applications to inhibit the growth of or kill fungi, which includes uses as disinfectants or as preservatives for materials such as foodstuffs, cosmetics, medicaments and other nutrient-containing materials.

In particular the present invention provides a method for the treatment and/or prevention of a fungal infection in a subject, comprising administering to a subject in need of such treatment and/or prevention at least one polyene macrolide derivative according the present invention, or a pharmaceutically acceptable salt or a pharmaceutical composition thereof, in therapeutically effective amounts. Typically the method for the treatment and/or prevention of a fungal infection comprises external and internal administration.

In a further aspect the invention provides a pharmaceutical composition comprising at least one polyene macrolide derivative according to the invention and a pharmaceutically acceptable carrier.

In yet another aspect the invention relates to the use of at least one polyene macrolide derivative of the present invention for preparing a medicament for the treatment and/or prevention of fungal infections in a subject.

In a further aspect, this invention also provides kits for use in exercising the methods of the present invention. For example, such kits may include at least one polyene macrolide derivative of the present invention and optional other pharmaceutically active agents or their pharmaceutical formulations in one or more vials.

### BRIEF DESCRIPTION OF FIGURES

FIG. 1. Native Amphotericin (1a, AmB), Nystatin (1b) and Pimaricin (1c)

FIG. 2. Schematic synthesis of N-doubly alkylated AmB: (a) P—(X₁)ₘ—(R₁)—CHO and P—(X₂)ₙ—(R₂)—CHO or (a′) P—(X₃)ₒ—(R₃)—(CHO); b) free acid: piperidine, DMSO, 95%; c) ester: piperidine, DMSO, then TMSCHN₂, R₄OH; d) amide R₄NH₂, PyBOP, HOBt, DIPEA, DMF, then piperidine, DMSO.

FIG. 3. K⁺ efflux from vesicles (LUVET₁₀₀) prepared from lapalmitoyl-2-oleoyl-glycero-3-phosphocholine (POPC) (solid line) or POPC with sterols (dotted line) for ergosterol and (broken line) for cholesterol caused by AmB (1a) (FIGS. 3a,b) and AmB diamine 3 (FIG. 3c,d), AmB diamine ester 9 (FIG. 3e,f) and AmB diamine amide 10 (FIG. 3g,h), added in DMSO to a suspension of vesicles to give final concentrations of 0.1 μM (FIG. 3a,c,e,g) and 1.0 μM (FIG. 3b,d,f,h).

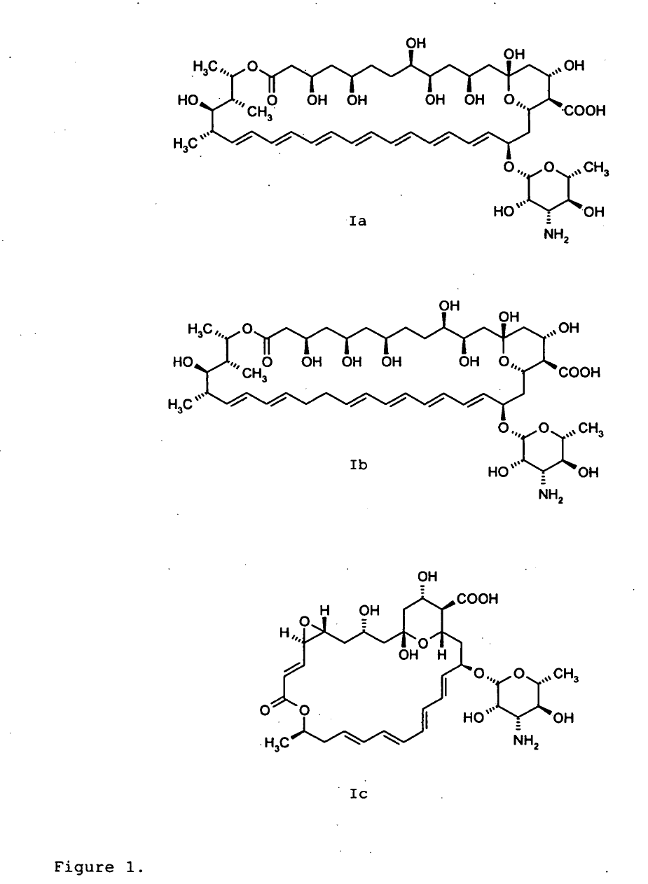

### DETAILED DESCRIPTION

Current treatment of fungal infections favor polyene macrolide antibiotics, which have proved to be the most effective antifungal agents due to their potent fungicidal activity, broad spectrum, and relatively low frequency of resistance among the fungal pathogens. Yet, the currently known polyene macrolides are also known for their high insolubility and high toxicity leading to serious side effects including renal failure, hypokalemia and thrombophlebitis. The present invention provides new polyene macrolide derivatives that provide significant advantages over the currently used polyene macrolides. Most importantly the polyene macrolide derivatives of the invention show low toxicity while retaining high antifungal activity as compared with amphotericin B (AmB). Furthermore the polyene macrolide derivatives of the invention can be designed such that they show an increased water solubility. Due to these favourable characteristics, the compounds of the invention need not require extensive formulations such as the lipid based formulations known for AmB and can thus be produced at low cost and allow easy storage and handling. The compounds of the invention showing these desirable characteristics are characterized by a polyene macrolide backbone having at least one free amino group, which is structurally modified double alkylation of the free amino group with at least one hydrocarbon group, wherein said at least one hydrocarbon group carries a total of at least two basic groups.

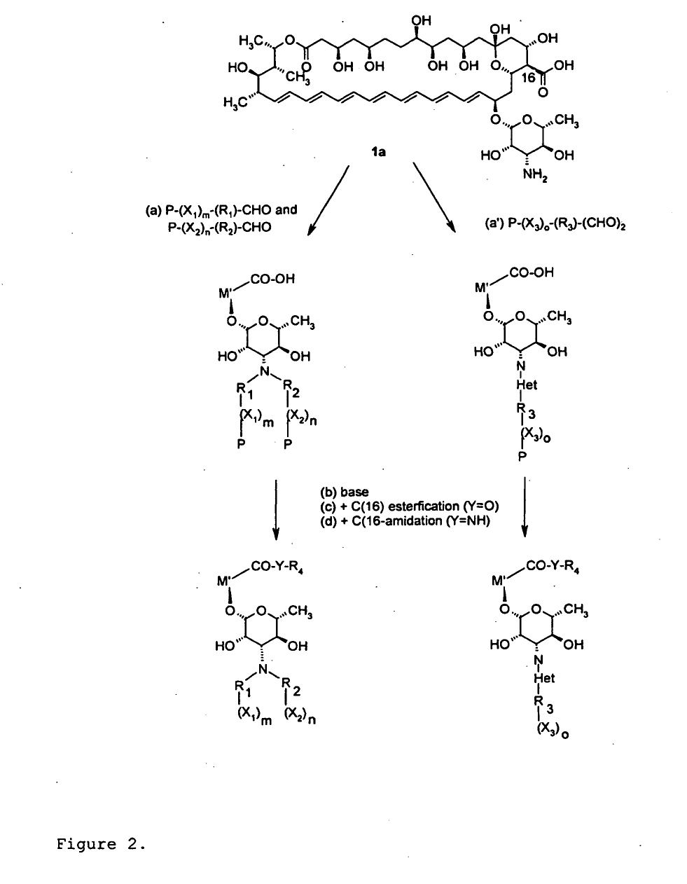

Double alkylation may be achieved with one single hydrocarbon group, resulting in a polyene macrolide derivative, wherein the amino group is embedded in a ring structure (FIG. 2, step (a′) ff). Alternatively, double alkylation may be achieved by two hydrocarbon groups, resulting in a polyene macrolide derivative, wherein the amino group is carrying two open chains (FIG. 2, step (a) ff). In one embodiment the at least two basic groups are present on one single hydrocarbon chain. In other embodiments the at least two basic groups are distributed among all hydrocarbon chains present. Thus in a specific embodiment double alkylation is performed with two hydrocarbon chains each carrying one or two basic groups. In another specific embodiment double alkylation is performed with one hydrocarbon chain carrying two, three or four basic groups. It is understood that the basic groups can be appended within or at the terminus of the hydrocarbon group. A skilled person will be aware of the different variants, which are all representative of the same inventive concept outlined hereinabove.

Thus, in a first aspect the present invention provides polyene macrolide derivatives of the invention which comprise a polyene macrolide backbone having at least one free amino group, wherein the amino group is doubly alkylated with at least one hydrocarbon group substituted with a total of at least two basic groups. In a specific embodiment a free carboxyl-substituent on the macrolide ring is optionally esterified or amidated.

Thus the present invention relates to polyene macrolide derivatives according to formula I:

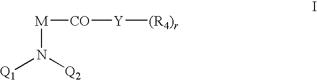

or a pharmaceutically acceptable salt thereof,
wherein:
M represents a polyene macrolide backbone;
Q₁ and Q₂ represent
   (i) a group of formula —(R₁)—(X₁)ₘ and —(R₂)—(X₂)ₙ, respectively, wherein
   X₁, X₂ represent independently of each other a basic group, preferably selected from —N(R₅)₂, —OH, —SH, —C(═NR₁₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
   m, n represent independently of each other 0, 1 or 2, with m+n≧2,
   R₁, R₂ represent independently of each other an unsubstituted or substituted hydrocarbon group, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; or
   (ii) taken together with the adjacent nitrogen atom to which they are attached, a nitrogen-containing heterocyclic group substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

The polyene macrolide backbone of the polyene macrolide derivatives of the invention may be derived form any known, or not yet known, but later discovered, polyene macrolide backbone having at least one free amino group. Typically, a polyene macrolide comprises a macrocyclic lactone ring with from four to seven conjugated double bonds (i.e. tetraenes, pentaenes, hexaenes, and heptaenes), in either an “all-trans” or “cis-trans” configuration, and various ketone and/or hydroxyl functions, e.g. a 1,3-polyhydroxyl system, and is glycosidically bound to a deoxysugar such as for example a mycosamine (3-amino 3,6 dideoxy-D-mannose) or perosamine (4-amino 4,6 dideoxy-D-mannose). Typical examples include amphotericin B, nystatin, candidin, candicidin, aureofacin, levorin, mycoheptin, partricin, perimycin, pimaricin, polyfungin, rimocidin or trichomycin, preferably amphotericin B, nystatin or pimaricin.

Thus in a specific embodiment the invention is directed to polyene macrolide derivatives according to formula II:

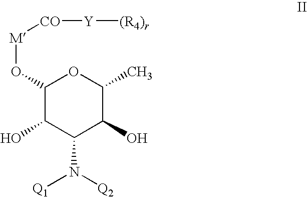

or a pharmaceutically acceptable salt thereof,
wherein:
M′ represents the macrocyclic lactone ring of a polyene macrolide backbone;
and Q₁, Q₂, Y, R₄, and r are as defined hereinabove.

In a further specific embodiment the invention is directed to polyene macrolide derivatives according to formulae III a-c:

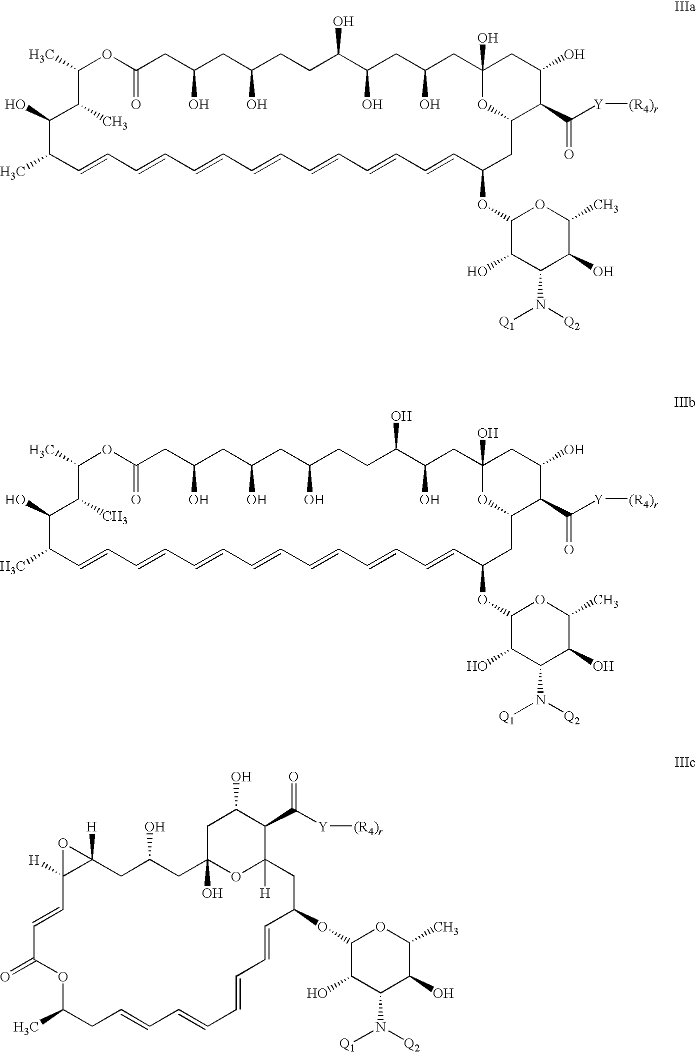

wherein:
Q₁, Q₂, Y, R₄, and r are as defined hereinabove.

The term “hydrocarbon group” should be understood to include preferably alkyl and cycloalkyl, in which one or more —CH₂-groups of the alkyl groups are optionally replaced by a group selected from —O—, —CO—, —COO—, —NR₅—, —NR₅CO—, —NR₅—COO—, —C(═NH)—NH—, —CH═CH— or —C≡C—, preferably —O—, —CO—, —COO—, —NR₅—, —NR₅CO—, —NR₅—COO—, —C(═NH)—NH—, wherein R₅ independently represents hydrogen or alkyl. As described herein before a hydrocarbon group is optionally substituted either within or at its terminus by one or more basic groups (X₁, X₂ or X₃). A skilled person will know which sites are accessible.

The term “alkyl” should be understood to include straight chain and branched alkyl groups having typically from 1 to 16, preferably from 1 to 10, more preferably from 1 to 6 carbon atoms, which may be optionally substituted with one or more substituents, which may be the same or different, and are selected from a group as defined hereinafter. Non-limiting examples of suitable alkyl groups include methyl, ethyl, n-propyl, isopropyl, n-butyl, t-butyl, n-pentyl, fluoromethyl and trifluoromethyl.

The term “branched” should be understood to represent a linear straight chain alkyl group having one or more lower alkyl groups, such as C(1-6) alkyl groups, such as methyl, ethyl or propyl, attached to it.

The term “alkoxy” should be understood to include “alkyl-O—”-groups, respectively wherein the alkyl groups are as described above. Methoxy, ethoxy and isopropoxy groups are especially preferred.

The term “cycloalkyl” refers to a saturated or unsaturated cyclic alkyl group having 3 to 10, preferably 3 to 6, more preferably 5 and 6 carbon atoms. Typical cycloalkyl groups include, but are not limited to, groups derived from cyclopropane, cyclobutane, cyclopentane, and cyclohexane.

The term “heterocycloalkyl” refers to cycloalkyl rings, wherein one or more of the cyclic alkyl groups is replaced by at least one heteroatom, for example 1 or 2 heteroatoms, selected from —O—, —NH— or —S—, preferably —O— and —NH—. Typical heterocycloalkyl groups include pyrrolidine, pyrazolidine, imidazolidine, tetrahydrofuran, piperidine, piperazine, and morpholine.

The term “aryl” should be understood to include an aromatic ring system having 5 to 14, preferably 5, 6, 9 or 10, more preferably 5 or 6 ring atoms. The aryl group can be substituted with one or more substituents, which may be the same or different, and are preferably selected from alkyl, —NH₂, —OH, —SH, —COR₅, —CSR₅, —COOR₅, —CONHR₅, —CN, or halogen, more preferably alkyl, —NH₂, —OH, —COOR₅, —CONHR₅, —CN, or halogen, wherein R₅ independently represents hydrogen or alkyl. Non-limiting examples of suitable aryl groups include phenyl, naphthalene, anthracene, phenanthrene or tetraline groups, preferably phenyl and napthyl groups, most preferably phenyl groups.

The term “heteroaryl” includes an “aryl” group as defined hereinabove comprising at least one heteroatom and thus should be understood to include an aromatic ring system of 5 to 14, preferably 5, 6, 9 or 10, more preferably 5 or 6 ring atoms, wherein one or more of the ring alkyl groups is replaced by at least one heteroatom, for example 1 or 2 heteroatoms, selected from —O—, —N—, —NH— or —S—, preferably —O—, —N— or —NH—. The heteroaryl can be optionally substituted by one or more substituents, which may be the same or different, and are preferably selected from alkyl, —NH₂, —OH, —SH, —COR₅, —CSR₅, —COOR₅, —CONHR₅, —CN, or halogen, more preferably alkyl, —NH₂, —OH, —COOR₅, —CONHR₅, —CN, or halogen, wherein R₅ independently represents hydrogen or alkyl. Non-limiting examples of suitable 6-membered heteroaryl groups include pyridine, pyrimidine, pyrazine, pyridazine and the like. Non-limiting examples of useful 5-membered heteroaryl rings include furan, thiophene, pyrrole, thiazole, isothiazole, imidazole, pyrazole, oxazole and isoxazole. Useful bicyclic groups are benzo-fused ring systems derived from the heteroaryl groups named above, e.g., quinoline, phthalazine, quinazoline, benzofuran, benzothiophene and indole. Preferred heteroaryl groups include pyridyl, pyrimidinyl, furyl, thienyl groups.

The term “arylalkyl” should be understood to include an aryl and an alkyl group as previously defined. Non-limiting preferred examples of suitable arylalkyl groups include benzyl, phenethyl and naphthyenylmethyl.

The term “heteroarylalkyl” should be understood to include a heteroaryl and an alkyl group as previously defined. Non-limiting examples of suitable heteroarylalkyl groups include e.g., pyridinylmethyl, pyrimidinylethyl and the like.

The term “nitrogen-containing heterocyclic group” refers to nitrogen-containing “heterocycloalkyl” and “aryl” groups and should be understood to include a saturated or unsaturated 4- to 10-membered, preferably 5- or 6-membered heterocyclic group, which is formed by Q₁, Q₂ and the adjacent nitrogen atom and which thus contains at least one nitrogen atom and may contain 1 to 3, preferably 1, further heteroatom, such as nitrogen, oxygen, sulphur, preferably nitrogen and oxygen, more preferably nitrogen, in addition to carbon atoms as the ring-constituting atoms. In a preferred embodiment a nitrogen-containing heterocyclic group includes a 5- or 6-membered heterocycloalkyl group, such as defined hereinabove. Most preferred examples of the nitrogen-containing heterocyclic group include six-membered rings, such as piperidine, 1-piperazine and the like. It is understood that the at least one substituent of formula —(R₃)—(X₃)ₒ may be attached to any of the available ring atoms of the nitrogen-containing heterocyclic group. In case of six membered rings, e.g. piperidine, the at least one substituent of formula —(R₃)—(X₃)ₒ may be attached at the 2-, 3-, 4-, 5- or 6-position, preferably at the 4-, 5- or 6-position, more preferably at the 6-position. It will be clear to a skilled person which sites are accessible.

The term “halogen” should be understood to include fluoro, chloro, bromo. iodo, preferably, fluoro and chloro, most preferably fluoro.

If not otherwise indicated, the term “unsubstituted or substituted” should be understood to represent optional substituents independently selected from the group consisting of alkyl, alkylene, cycloalkyl, aryl, heteroaryl, arylalkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halogen, nitro, cyano, carboxy, alkoxycarbonyl, aryloxycarbonyl, aralkoxycarbonyl, aminoalkyl, alkylthio, arylthio, heteroarylthio, aralkylthio, heteroaralkylthio, cycloalkyl, cycloalkenyl, heterocyclyl, heterocyclenyl, preferably alkyl, hydroxy, alkoxy, cyano, amino, —NH(alkyl), —N(alkyl)₂ (which alkyls can be the same or different), carboxy, —C(O)O-(alkyl) and halogen. Those skilled in the art will recognize that the size and nature of the substituent(s) will affect the number of substituents which can be present.

In a specific embodiment Y is O, N, or NH.

In another specific embodiment X₁, X₂ and X₃ represent independently of each other a basic group selected from —NHR₅, —OH, —C(═NH)—NHR₅, —NH—C(═NH)—NHR₅, —N₃, —COR₅, —COOR₅, and —CONHR₅, wherein R₅ represents hydrogen or C(1-10)alkyl, more preferably a basic group selected from —NH₂, —OH, —C(═NH)—NH₂, —NH—C(═NH)—NH₂, —COOR₅, and —CONHR₅, wherein R₅ represents hydrogen or C(1-10)alkyl.

In a further embodiment X₁ and X₂ are the same.

In a specific embodiment R₁, R₂ and R₃ represent linear or branched C(1-10)alkyl, C(4-10)cycloalkyl or C(4-10)heterocycloalkyl, which are unsubstituted or substituted by —NH₂, —OH, —COOR₅, —CONHR₅, or —CN, and in which one or more —CH₂-groups of the alkyl groups are optionally replaced by —O—, —CO—, —COO—, —NR₅—, —NR₅CO—, —C(═NH)—NH—, —CH═CH—, wherein R₅ independently represents hydrogen or alkyl. It is understood, that only —CH₂— groups of alkyl groups non-adjacent to the N-atom can be replaced in order to obtain N-doubly alkylated derivatives.

In another specific embodiment R₁, R₂, and R₃ represent independently of each other linear or branched C(1-10)alkyl, C(4-10)cycloalkyl or C(4-10)heterocycloalkyl, which are unsubstituted or substituted by —NH₂, —OH, —COOR₅, —CONHR₅, or alkyl, and in which one or more —CH₂— groups of the alkyl groups are optionally replaced by —O—, —CO—, —COO—, —CONR₅—, —NR₅—, —CH═CH—, wherein R₅ independently represents hydrogen or alkyl.

In a further embodiment R₁ and R₂ are the same.

In a specific embodiment R₄ represents hydrogen, C(1-10)alkyl, C(4-10)cycloalkyl or C(4-10)heterocycloalkyl, which is unsubstituted or substituted by —NH₂, —OH, —COOR₅, —CONHR₅, —CN, halogen or alkyl, preferably —NH₂, —OH, halogen or alkyl, and in which one or more —CH₂— groups are optionally replaced by —O—, —CO—, —COO—, —CH═CH—, preferably —O—, —COO—.

Thus in a further embodiment the present invention is directed to compounds of formula I

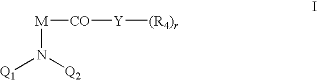

or a pharmaceutically acceptable salt thereof,
wherein:
M represents a polyene macrolide backbone;
Q₁ and Q₂ represent
   (i) a group of formula —(R₁)—(X₁)ₘ and —(R₂)—(X₂)ₙ, respectively, wherein
   X₁, X₂ represent independently of each other a basic group selected from —NHR₅, —OH, —C(═NH)—NHR₅, —NH—C(═NH)—NHR₅, —N₃, —COR₅, —COOR₅, and —CONHR₅, wherein R₅ represents hydrogen or C(1-10)alkyl;
   m, n represent independently of each other 0, 1 or 2, with m+n≧2,
   R₁, R₂ represent independently of each linear or branched C(1-10)alkyl, C(4-10)cycloalkyl or C(4-10)heterocycloalkyl, which are unsubstituted or substituted by —NH₂, —OH, —COOR₅, —CONHR₅, or alkyl, and in which one or more —CH₂— groups of the alkyl groups are optionally replaced by —O—, —CO—, —COO—, —CH═CH—, wherein R₅ independently represents hydrogen or alkyl; or
   (ii) taken together with the adjacent nitrogen atom to which they are attached, a 6-membered heterocyclic group containing from 1 to 3 heteroatoms, which is substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, N or NH,
R₄ represents hydrogen, C(1-10)alkyl, C(4-10)cycloalkyl or C(4-10)heterocycloalkyl, which is unsubstituted or substituted by —NH₂, —OH, —COOR₅, —CONHR₅, —CN, halogen or alkyl, preferably —NH₂, —OH, halogen or alkyl, and in which one or more —CH₂— groups are optionally replaced by —O—, —CO—, —COO—, —CH═CH—, preferably —O—, —COO—, wherein R₅ independently represents hydrogen or alkyl; and
r is 1 or 2.

In a specific embodiment R₁ and R₂ are the same.

In a further embodiment X₁ and X₂ are the same.

In further embodiments the invention is also directed to polyene macrolide derivatives according to formula II (with M′ being a macrocyclic lactone ring of a polyene macrolide backbone) or formulas III a-c or a pharmaceutically acceptable salt thereof, wherein:
Q₁ and Q₂ represent
   (i) a group of formula —(R₁)—(X₁)ₘ and —(R₂)—(X₂)ₙ, respectively, wherein
   X₁, X₂ represent independently of each other a basic group selected from —NHR₅, —OH, —C(═NH)—NHR₅, —NH—C(═NH)—NHR₅, —N₃, —COR₅, —COOR₅, and —CONHR₅, wherein R₅ represents hydrogen or C(1-10)alkyl;
   m, n represent independently of each other 0, 1 or 2, with m+n≧2,
   R₁, R₂ represent independently of each linear or branched C(1-10)alkyl, C(4-10)cycloalkyl or C(4-10)heterocycloalkyl, which are unsubstituted or substituted by —NH₂, —OH, —COOR₅, —CONHR₅, or alkyl, and in which one or more —CH₂— groups of the alkyl groups are optionally replaced by —O—, —CO—, —COO—, —CH═CH—, wherein R₅ independently represents hydrogen or alkyl; or
   (ii) taken together with the adjacent nitrogen atom to which they are attached, a 6-membered heterocyclic group containing from 1 to 3 heteroatoms, which is substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, N or NH,
R₄ represents hydrogen, C(1-10)alkyl, C(4-10)cycloalkyl or C(4-10)heterocycloalkyl, which is unsubstituted or substituted by —NH₂, —OH, —COOR₅, —CONHR₅, —CN, halogen or alkyl, preferably —NH₂, —OH, halogen or alkyl, and in which one or more —CH₂— groups are optionally replaced by —O—, —CO—, —COO—, —CH═CH—, preferably —O—, —COO—, wherein R₅ independently represents hydrogen or alkyl; and
r is 1 or 2.

In a specific embodiment R₁ and R₂ are the same.

In a further embodiment X₁ and X₂ are the same.

In a further aspect, the present invention is directed to a specific subgroup of polyene macrolide derivatives according to formula IV:

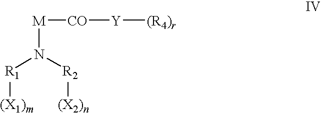

or a pharmaceutically acceptable salt thereof,
wherein:
M represents a polyene macrolide backbone;
R₁, R₂ represent independently of each other linear or branched —(CH₂)ₚ—, wherein p is an integer from 0 to 12 and in which one or more —CH₂— groups are optionally replaced by —O—, —CO—, —COO—, —CONR₅—, —NR₅—, —CH═CH—, wherein R₅ independently represents hydrogen or alkyl;
X₁, X₂ represent independently of each other a basic group, which may be attached to any —CH₂— group of R₁ and R₂, respectively, and is preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

In a particular embodiment, the present invention is directed to polyene macrolide derivatives according to formula IV or a pharmaceutically acceptable salt thereof,

wherein:
M represents a polyene macrolide backbone;
R₁, R₂ represent independently of each other linear or branched —(CH₂)ₚ—, wherein p is an integer from 0 to 12;
X₁, X₂ represent independently of each other a basic group, which may be attached to any —CH₂— group of R₁ and R₂, respectively, and is selected from —NHR₅, —OH, —C(═NH)—NHR₅, —NH—C(═NH)—NHR₅, —N₃, —COR₅, —COOR₅, and —CONHR₅, wherein R₅ represents hydrogen or alkyl;
m, n represent independently of each other 1 or 2;
Y represents O, N or NH;
R₄ represents hydrogen, C(1-10)alkyl, C(4-10)cycloalkyl or C(4-10)heterocycloalkyl, which is unsubstituted or substituted by —NH₂, —OH, —COOR₅, —CONHR₅, —CN, halogen or alkyl, preferably —NH₂, —OH, halogen or alkyl, and in which one or more —CH₂— groups are optionally replaced by —O—, —CO—, —COO—, —CH═CH—, preferably —O—, —COO—, wherein R₅ independently represents hydrogen or alkyl; and; and
r is 1 or 2.

In a specific embodiment R₁ and R₂ are the same.

In a further embodiment X₁ and X₂ are the same.

Specifically the present invention is directed to polyene macrolide derivatives according to formula V:

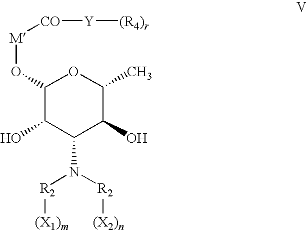

or a pharmaceutically acceptable salt thereof,
wherein:
M′ represents the macrocyclic lactone ring of a polyene macrolide backbone;
and R₁, R₂, R₄, X₁ and X₂, Y, m, n, and r are as defined hereinabove.

In a specific embodiment R₁ and R₂ are the same.

In a further embodiment X₁ and X₂ are the same.

More specifically the present invention is directed to polyene macrolide derivatives according to formulae VIa-e or a pharmaceutically acceptable salt thereof,

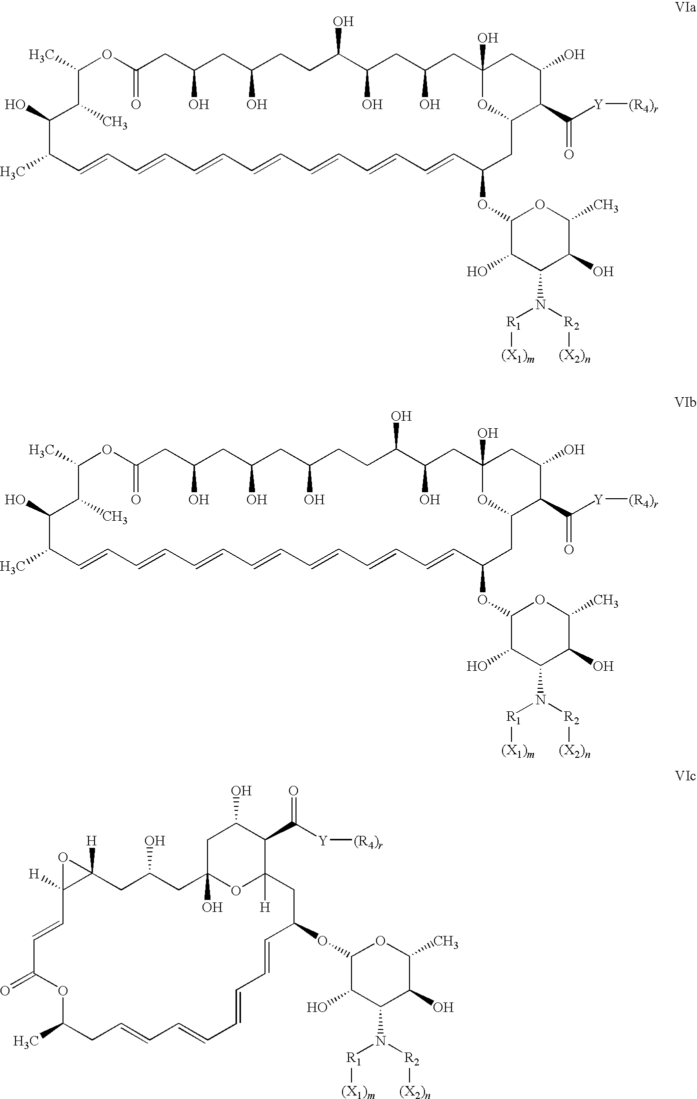

wherein:
R₁, R₂, R₄, X₁ and X₂, Y, m, n, and r are as defined hereinabove.

In a specific embodiment p is an integer from 0 to 10, more specifically 0 to 6.

In a specific embodiment R₁ and R₂ are the same.

In a further embodiment X₁ and X₂ are the same.

In a further aspect, the present invention is directed to a further subgroup of the polyene macrolide derivatives of the present invention according to formula VII:

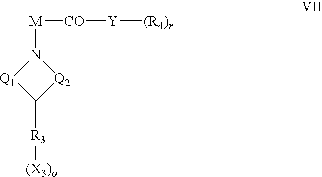

or a pharmaceutically acceptable salt thereof,
wherein:
M represents a polyene macrolide backbone;
Q₁, Q₂ form together with the adjacent nitrogen atom to which they are attached a nitrogen-containing heterocyclic group;
X₃ represents a basic group, which may be attached to any —CH₂-group of R₃, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

In a further specific embodiment the present invention is directed to polyene macrolide derivatives according to formula VIII:

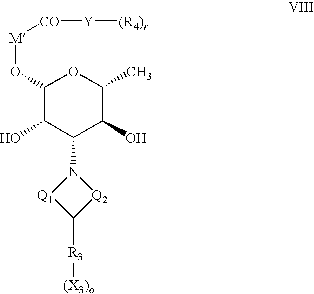

or a pharmaceutically acceptable salt thereof,
wherein:
M′ represents the macrocyclic lactone ring of a polyene macrolide backbone;
and Q₁, Q₂, R₃, R₄, X₃, Y, O, and r are as defined hereinabove.

More specifically the present invention is directed to polyene macrolide derivatives according to formulae IX a-c or a pharmaceutically acceptable salt thereof,

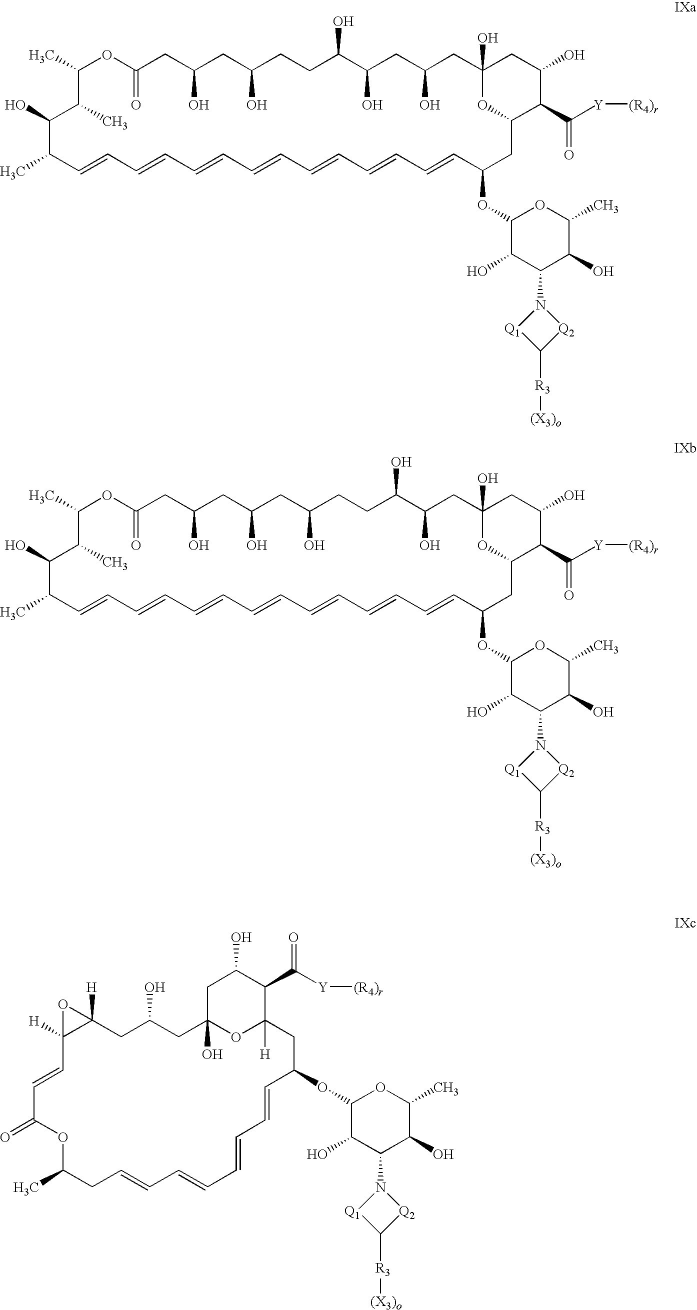

wherein:
Q₁, Q₂, R₃, R₄, X₃, Y, o, and r are as defined hereinabove.

In a further specific embodiment the present invention relates to polyene macrolide derivative of formula X:

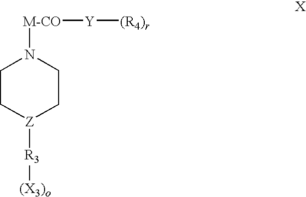

or a pharmaceutically acceptable salt thereof,
wherein:
M represents a polyene macrolide backbone;
Z represents —CH— or —N—;
X₃ represents a basic group, which may be attached to any —CH₂-group of R₃, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

Specifically the present invention is directed to polyene macrolide derivatives according to formula XI:

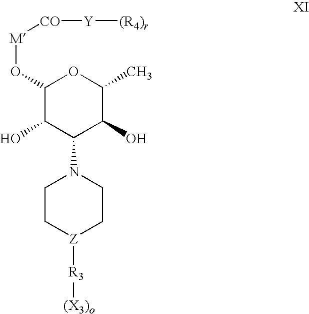

or a pharmaceutically acceptable salt thereof,
wherein:
M′ represents the macrocyclic lactone ring of a polyene macrolide backbone;
and R₃, R₄, X₃, Z, Y, o and r are as defined hereinabove.

More specifically the present invention is directed to polyene macrolide derivatives according to formulae XII a-c or a pharmaceutically acceptable salt thereof,

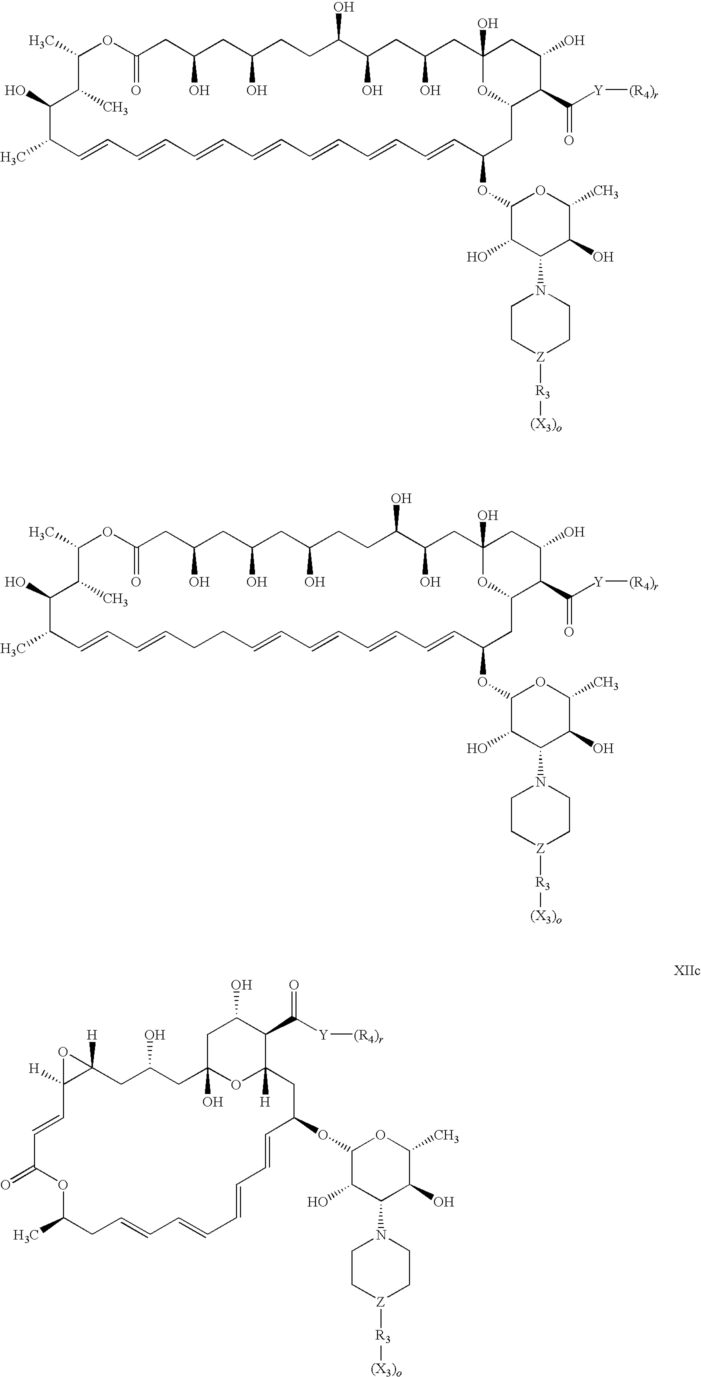

wherein:
R₃, R₄, X₃, Z, Y, o and r are as defined hereinabove.

Those of skill in the art will appreciate that the compounds of the invention described the formulas herein above and their specific embodiments are not intended as limiting and are intended to encompass all possible tautomeric, conformational isomeric, enantiomeric or geometric isomeric forms, including which will be shown to have antifungal activity at a later date.

In a further aspect the invention relates to a method of producing a polyene macrolide derivative according to the invention, comprising subjecting a polyene macrolide to double reductive alkylation using standard chemistry protocols. More specifically, a polyene macrolide may be subjected to double reductive alkylation with two optionally protected functionalized aldehydes P—(X₁)ₘ—(R₁)—CHO and P—(X₂)ₙ—(R₂)—CHO, wherein X₁, X₂, R₁ and R₂ are as defined hereinabove and P is H or a suitable protecting group, to give a compound of the invention wherein the amino group is alkylated with two open chains. Alternatively a polyene macrolide may be subjected to double reductive alkylation with one optionally protected functionalized aldehyde P—(X₃)ₙ—(R₃)—(CHO)₂, wherein X₃ and R₃ are as defined hereinabove and P is H or a suitable protecting group, to give a compound of the invention wherein the amino group is embedded in a ring structure.

The above double reductive alkylation protocol provides the opportunity to readily access a wide variety of polyene macrolide derivatives according to the invention. FIG. 2 illustrates a typical example, wherein the synthesis commences with the reaction of AmB (1a) with P—(X₁)ₘ—(R₁)—CHO and P—(X₂)ₙ—(R₂)—CHO (step (a)) or P—(X₃)ₒ—(R₃)—(CHO)₂ (step (a′)) to give the corresponding N-protected derivative. This may conveniently be carried out on native AmB (1a), without protecting groups, to deliver the N-protected intermediate as a common precursor for the preparation of various derivatives. Subsequent treatment of the N-protected intermediate with a base, e.g. piperidine furnishes the free amine (FIG. 2, step b), which may optionally be subjected to esterification at the C-16 position to obtain the corresponding ester (FIG. 2, step c). Alternatively, the corresponding C-16 amide may be prepared via an amide coupling reaction according to known chemistry protocols (FIG. 2, step d).

The synthetic route according to FIG. 2 allows to perform the synthesis of the polyene macrolide derivatives either in a one-pot reaction or else the intermediates may be isolated and/or purified using standard techniques, such as crystallization, precipitation and/or chromatography (normal reverse phase, and preparative HPLC).

It is understood that other known synthetic routes can be employed to arrive at the compounds of the invention.

The polyene macrolide derivatives of the present invention have significant advantages over currently available polyene macrolide antifungals. Specifically, the polyene macrolide derivatives of the present invention show excellent therapeutic potency, typically having minimum inhibitory concentrations (MICs) of as low as 0.1 μM or less against e.g. Saccaromyces cerevisiae in standard in vitro assays (Table 1 and Examples section), i.e. having a MIC of at least 15 times less than AmB. Furthermore the polyene macrolide derivatives also showed a dramatic increase in antifungal activity against AmB resistant strains, e.g. having minimum inhibitory concentrations (MICs) of as low as 1 μM against e.g. Candida albicans in standard in vitro assays (Table 1 and Examples section). Thus typically the compounds of the invention will exhibit MICs of less than about 10 μM, usually less than about 5 μM, preferably less than about 1 μM against Saccaromyces cerevisiae, Candida albicans etc. using standard methods. Clearly compounds having lower MICs are preferred. Since the mechanism of action is believed to be, at least in part, dependent upon binding to a sterol moiety, such as ergosterol, present in the membrane, the activity of the polyene macrolide derivatives of the present invention was assessed in a further assay based on K⁺ efflux measurements from sterol-containing vesicles. Both efficient ion channel formation and good selectivity was observed. Generally, active polyene macrolide derivatives of the invention and their antifungal activity are identified using in vitro screening assays, such as the ones used herein, that are well-known in the art. It will be apparent to a skilled person, that alternatively, the polyene macrolide derivatives of the invention may also be assessed for antifungal activity using an assay based on in vivo models or other assays, that are well known in the art or that will become apparent to those skilled in the art upon review of this disclosure.

Thus in yet another aspect the present invention provides methods of inhibiting the growth of fungi, such as Candida species (e.g. C. albicans, C. glabrata), Saccharomyces cerevisiae, Aspergillus species, Crytococcusneoformans, Blastomyces dennatitidis, Histoplasmacapsulatum, Torulopsis glabrata, Coccidioidesimmitus, Paracoccidioides braziliensis, and the like, which methods comprise contacting a fungus with an effective amount of a polyene macrolide derivative of the invention, or a pharmaceutically acceptable salt thereof, or a pharmaceutical composition thereof to inhibit the growth of the fungus.

In addition, the polyene macrolide derivatives of the present invention show higher water solubility as compared to AmB, and significantly reduced hemotoxicity, i.e. a low degree of hemolysis of human erythrocytes (see Examples section). As discussed in detail hereinafter, all of the polyene macrolide derivatives of the invention may be administered externally, e.g. topically, or internally, e.g. systemically. For in vivo applications, such as for systemic administration and/or for use in treating or preventing systemic infections, compounds that exhibit significant antifungal activity, higher water-solubility than AmB (at approx. neutral pH) and low toxicity are preferred. For topical administration and applications water solubility is a particular concern.

Thus in view of their superior characteristics, the present invention provides in a further aspect polyene macrolide derivatives according to the invention (including pharmaceutically acceptable salts and/or pharmaceutical compositions thereof) for use in therapy, in particular for use in the treatment and/or prevention of fungal infections.

Other uses include further applications to inhibit the growth of or kill fungi, which includes uses as disinfectants or as preservatives for materials such as foodstuffs, cosmetics, medicaments and other nutrient-containing materials.

In particular the present invention provides a method for the treatment and/or prevention of a fungal infection in a subject, comprising administering to a subject in need of such treatment and/or prevention at least one polyene macrolide derivative according the present invention, or a pharmaceutically acceptable salt or a pharmaceutical composition thereof, in therapeutically effective amounts. Typically the method for the treatment and/or prevention of a fungal infection comprises external and internal administration. The mode of administration will depend upon the nature of the infection. Thus the compounds of the invention may be formulated for intravenous, intra peritoneal, oral, topical, subcutaneous, rectal or vaginal administration as described hereinafter.

The subject in need of such treatment and/or prevention according to the present invention is preferably a mammalian subject, i.e. an animal or human, preferably a human.

When used to treat or prevent fungal infections the polyene macrolide derivatives of the invention can be administered or applied singly, as mixtures of two or more polyene macrolide derivatives, in combination with one or more other antifungal, antibiotic or antimicrobial agents or in combination with other pharmaceutically active agents. The polyene macrolide derivatives can be administered or applied per se or as pharmaceutical compositions. The specific pharmaceutical formulation will depend upon the desired mode of administration, and will be apparent to those having skill in the art. Numerous compositions for the topical or systemic administration of polyene macrolides are described in the literature. Any of these compositions may be formulated with the polyene macrolide derivatives of the invention.

Thus, in a further aspect the invention provides a pharmaceutical composition comprising at least one polyene macrolide derivative according to the invention and a pharmaceutically acceptable carrier.

In yet another aspect the invention relates to the use of at least one polyene macrolide derivative of the present invention for preparing a medicament for the treatment and/or prevention of fungal infections in a subject.

Pharmaceutical compositions comprising the polyene macrolide derivatives of the invention may be manufactured by means of conventional mixing, dissolving, granulating, tabletting, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active polyene macrolide derivatives into preparations which can be used pharmaceutically. As mentioned hereinbefore proper formulation is dependent upon the route of administration chosen.

For topical administration the polyene macrolide derivatives of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the polyene macrolide derivatives of the invention may be formulated in aqueous solutions, preferably in physiologically compatible buffers such as Hanks's solution, Ringer's solution, or physiological saline buffer. The solution may contain further additives such as suspending, stabilizing and/or dispersing agents.

Alternatively, the polyene macrolide derivatives may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the polyene macrolide derivatives can be readily formulated by combining them with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject in need for treatment. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc.

Additionally, flavoring agents, preservatives, coloring agents and the like may be added.

For buccal administration, the compositions may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described hereinabove, the polyene macrolide derivatives may also be formulated as a depot preparation. Such long acting formulations may be administered by implantation (for example subcutaneously or intramuscularly) or by intramuscular injection. Thus, for example, the compounds may be formulated with suitable polymeric or hydrophobic-materials (for example as an emulsion in an acceptable oil) or ion exchange resins, or as sparingly soluble derivatives, for example, as a sparingly soluble salt.

Alternatively, other pharmaceutical delivery systems including lipid-based formulations and emulsions may be used to deliver the polyene macrolide derivatives of the invention. Additionally, the polyene macrolide derivatives may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days.

Depending on the substitution pattern of the polyene macrolide derivatives of the invention, the derivatives may be in form of esters, amides as well as free acids, free bases or as pharmaceutically acceptable salts. Pharmaceutically acceptable salts are those salts which retain substantially the antifungal activity of the free acids or bases and which are prepared by reaction with bases or acids, respectively. Pharmaceutical salts tend to be more soluble in aqueous and other protic solvents than are the corresponding free base or acid forms. Some examples of pharmaceutically acceptable salts include: (1) acid addition salts, formed with inorganic acids such as hydrochloric acid, hydrobromic acid, sulfuric acid, nitric acid, phosphoric acid, and the like; or formed with organic acids such as amino acids (e.g., aspartic acid, glutamic acid, asparagine, glutamine, lysine, ornithine) acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the derivative is either replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In one embodiment, pharmaceutically acceptable salts are formed with aspartic acid, glutamic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid and mandelic acid. In another embodiment, pharmaceutically acceptable salts are formed with aspartic acid, glutamic acid, and fumaric acid.

The polyene macrolide derivatives of the invention, or pharmaceutical compositions thereof, will typically be used in an amount effective to achieve the intended purpose. It is understood that the actual amount used will depend on the particular application, such as treatment and/or prevention of fungal infections or use as a disinfectant or preservative, the subject to be treated and the route of administration.

For example, for use as a disinfectant or preservative, an antifungally effective amount of a polyene macrolide derivative, or composition thereof, is applied or added to the material to be disinfected or preserved. By antifungally effective amount is meant an amount of polyene macrolide derivative or composition that inhibits the growth of, or is lethal to, a target fungi.

While the actual amount will depend on a particular target fungi and application, for use as a disinfectant or preservative the polyene macrolide derivatives, or compositions thereof, are usually added or applied to the material to be disinfected or preserved in relatively low amounts. Typically, the polyene macrolide derivative comprises less than about 5% by weight of the disinfectant solution or material to be preserved, preferably less than about 1% by weight and more preferably less than about 0.1% by weight. An ordinarily skilled artisan will be able to determine antifungally effective amounts of particular polyene macrolide derivatives for particular applications without undue experimentation using, for example, the in vitro assays provided in the examples.

For use in the treatment and/or prevention of fungal infections, the polyene macrolide derivatives of the invention, or compositions thereof, are administered or applied in a therapeutically effective amount. By therapeutically effective amount is meant an amount sufficient to achieve the desired effect, which is to ameliorate the symptoms of, treat or prevent fungal infections.

Determination of a therapeutically effective amount is well within the capabilities of those skilled in the art, especially in light of the detailed disclosure provided herein.

For topical administration to treat or prevent fungal infections, a therapeutically effective dose can be determined using, for example, the in vitro assays provided in the examples. The treatment may be applied while the infection is visible, or even when it is not visible. An ordinarily skilled artisan will be able to determine therapeutically effective amounts to treat topical infections without undue experimentation.

For systemic administration, a therapeutically effective dose can be estimated initially from in vitro assays. For example, a dose can be formulated in animal models to achieve a circulating polyene macrolide derivative concentration range that includes the MIC as determined in cell culture. Such information can be used to more accurately determine useful doses in humans.

Initial dosages can also be estimated from in vivo data, e.g., animal models, using techniques that are well known in the art. One having ordinary skill in the art can readily optimize administration to humans based on animal data.

Alternatively, initial dosages can be determined from the dosages administered of known polyene macrolides (e.g., AmB) by comparing the MIC of the specific polyene macrolide derivative with that of a known polyene macrolide, and adjusting the initial dosages accordingly. The optimal dosage may be obtained from these initial values by routine optimization.

Dosage amount and interval may be adjusted individually to provide plasma levels of the active polyene macrolide derivative which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering a single daily dose or multiple doses each day.

The low toxicity of the polyene macrolide derivatives compared to AmB allows any known administration including an administration in a manner similar to AmB. Typical dosages and routes of administration used for AmB are well-known (see, e.g., Goodman and Gilman's The Pharmacological Basis of Therapeutics, 8 Edition, 1990, Pergamon Press Inc., pp. 1165-1168, incorporated herein by reference). The administration may be part of a continuous treatment or may be repeated intermittently to treat existing infections or alternatively may be part of a preventive antifungal therapy, e.g. when infections are not detectable.

The amount of polyene macrolide derivative administered will, of course, be dependent on, among other factors, the subject being treated, the subject's weight, the general health condition of the subject, the severity of the infection, the manner of administration, e.g. systemic or local, oral or intravenous, etc., as well as the judgment of the prescribing physician. For example, in cases of local administration or selective uptake, the effective local concentration of polyene macrolide derivative may not be related to plasma concentration.

The compounds of the invention may be part of a monotherapy, i.e. administered alone, or part of a combination therapy with one or more compounds of the invention or one or more other pharmaceutically active agents, such as for example other antifungals, antibiotics or antimicrobials.

The skilled person will be able to optimize therapeutically effective dosages without undue experimentation. Preferably, a therapeutically effective dose of the polyene macrolide derivatives of the invention will provide therapeutic benefit without causing substantial toxicity. Toxicity of the polyene macrolide derivatives can be determined using standard pharmaceutical procedures in cell cultures or experimental animals, e.g., by determining the EH₅₀ value (toxicity towards human erythrocytes) or LD₅₀ (the dose lethal to 50% of the population). The dose ratio between toxic and therapeutic effect is the therapeutic index. Polyene macrolide derivatives which exhibit high therapeutic indices are preferred. The data obtained from these cell culture assays and animal studies can be used in formulating a dosage range that is not toxic for use in human. The dosage of the polyene macrolide derivatives described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. See, e.g., Fingl and Woodbury, “Chapter 1: General Principles,” in “The Pharmacological Basis of Therapeutics”, 5th ed, Goodman and Gilman eds, MacMillan Publishing Co., Inc., New York, pp. 1-46 (1975).

In a further aspect, this invention also provides kits for use in exercising the methods of the present invention. For example, such kits may include at least one polyene macrolide derivative of the present invention and optional other pharmaceutically active agents or their pharmaceutical formulations in one or more vials. For example, in certain embodiments, the kit may include a single pharmaceutical composition, present as one or more unit dosages, comprising at least one polyene macrolide derivative of the present invention. In yet other embodiments, the kit may include two or more separate pharmaceutical compositions, each containing either polyene macrolide derivative of the present invention or another pharmaceutically active agent, such as for example other antifungals, antibiotics or antimicrobials. In addition to the above components, the kits may further include instructions for practicing the methods of this invention.

The invention having been described, the following examples are presented to illustrate, rather than to limit, the scope of the invention. It is understood that any modifications and methods which are functionally equivalent and which will become apparent to those skilled in the art from the foregoing description and figures are intended to fall within the scope of the invention.

### EXAMPLES

### Chemical Syntheses

Materials and Methods. All reactions were carried out in oven-dried glassware under an atmosphere of argon. Amphotericin B (AmB) was purchased from Apollo Scientifics (90% HPLC purity). Nystatin dihydrate (Mycostatin or Fungicidin) was purchased from Applichem (95% HPLC purity). Pimaricin was purchased from Aldrich (95% HPLC purity). All the other compounds were purchased from Fluka, Senn and Aldrich and used without further purification. Dimethylformamide was purified by distillation and methanol was distilled over magnesium oxide. Diisopropylethylamine and piperidine were distilled from KOH under nitrogen. The reactions were monitored by thin layer chromatography using Merck Silica Gel 60 FB254B plates and visualized using UV and aqueous ceric ammonium molybdate stain. Flash chromatography was performed using E. Merck Silica Gel 60 (230-400 mesh). UV-VIS spectra were recorded with a Varian Cary 50 Conc UV-Visible Spectrophotometer. The NMR spectra were recorded on a Bruker DRX-500 spectrometer (500 MHz). Chemical shifts (δ) are reported in ppm with the tetramethylsilane resonance as the internal standard (δ 0.00) for ¹H and ¹³C. The data are reported as follows: (s=singlet, d=doublet, t=triplet, q=quartet, m=multiplet or unresolved, br=broad signal, coupling constant in Hz, integration). ¹³C NMR spectra were recorded with complete proton decoupling. Mass spectrometric measurements were performed on a Bruker Reflex MALDI-TOF using 2,5-dihydroxy benzoic acid as matrix (20 kV).

### Biological Assays

In Vitro Antifungal Activity. MIC values for various strains were determined in accordance with the standard protocol from the National Committee of Clinical Laboratory Standards but using YEPD liquid medium instead of RPMI-1640 medium (National Committee of Clinical Laboratory Standards (Reference Method for Broth Dilution Antifungal Susceptibility Testing of Yeast, M27-A2, Approved Standard-Second Edition, Volume 22, Number 15, 1995). AmB was used as a reference. The strains were cultivated overnight in 5 mL YEPD liquid medium at 30 to 37° C. (30° C. for the strain BY4741; 37° C. for the strain DSY1764) with constant shaking. The saturated cultures were diluted to an ODB₆₀₀ of 0.1 (3×10P⁷ cells/mL). Using 24 wells plate, each well was prepared by adding 1% (12 μL) of DMSO solution of the tested polyene macrolide with 1% (12 μL) yeast cells solution and completed with YEPD (1.176 mL). The plates were sealed with Parafilm and then incubated for 18 to 36 hours at 30 to 37° C. (18 hours at 30° C. for the strain BY4741; 36 hours at 37° C. for the strain DSY1764). The optical density was read at 600 nm using 1.5 mL cuvettes. The MIC value was defined as the drug concentration needed to inhibit growth, less than 5% compared to a drug-free culture.

Hemolytic Assay. The hemotoxicity of the polyene macrolide derivatives of the invention was determined by measuring the toxicity towards human erythrocytes (EH₅₀ value) using an established assay (Kinsky, S. C. et al., Biochem. Biophys. Res. Comm. 1962, 9, 503). Human blood, anticoagulated with citrate or EDTA, was centrifuged (2000×g) at 4° C. for 10 minutes. The pellets were washed three times with PBS buffer (pH 7.2 with 2 g/L glucose) and then diluted to a concentration of 4% (4×10⁸ cells/mL). All experiments were done in triplo and the total volume for the hemolysis tubes was 1.4 ml. The solutions were prepared by adding 1% (14 μL) of DMSO solution of the tested compound with 736 μL of PBS buffer and completed with 750 μL of 4% erythrocytes. After one hour incubation at 37° C., the samples were centrifuged (1500×g) at 4° C. for 5 min and the absorbance of the supernatant was measured at 560 nm. The concentration that led to 50% hemolysis (EH₅₀) was intrapolated graphically. The value for the 100% hemolysis was obtained by the treatment with 100 μM of AmB.

K+ efflux assay. The K⁺ efflux measurements were made following a previously described procedure (Zumbuehl, A. et al., Angew. Chem. Int. Ed. 2004, 43, 5181-5185; Zumbuehl, A. et al., Org. Lett. 2004, 6, 3683-3686). The appropriate liposome suspensions were prepared using POPC, cholesterol and ergosterol. Then the liposomes were sized by extrusion through two 400 nm, 200 nm, and finally 100 nm pore size membrane. The resultant “100 nm” unilamellar liposomes were dialyzed against 600 mL of 150 mM NaCl, 5 mM HEPES (pH 7.4) buffer. After, the suspension was diluted with 150 mM NaCl, 5 mM HEPES (pH 7.4) buffer to 1 mM overall lipid concentration (phospholipids+sterols). For each efflux measurement 10 mL of this liposome suspension was placed in a small beaker. Potentiometric measurements were performed with a 16-channel electrode monitor in magnetically stirred solutions at ambient temperature. The reference electrode was a Metrohm double junction Ag/AgCl reference electrode with 3 M KCl as reference electrolyte and 1 M LiOAc as bridge electrolyte. After recording the amphotericin-induced potassium efflux, liposomes were lysed by adding sodium cholate (172 mg). The resulting reading (taken after 0.5 h) was used to quantify the 100% K+-release.

Solubility assay. The solubility assay was performed according to Lipinski, C. A. et al Advances Drug Delivery Reviews 2001, 46, 3-26. Typically, a compound of the invention was dissolved in DMSO at a concentration of 10 μg/μl. The so obtained solution was added in 1 ml aliquots at 1 min intervals to 2.5 ml PBS buffer solution pH 7.2. These additions correspond to solubility increments of 5 μg/ml to a top value of 65 μg/ml. A total of 14 additions were made and each time increased UV absorbance from light scattering was measured at 600 nm. The precipitation point was calculated from a bilinear curve fit to the Absorbance (y axis) versus μl of DMSO (x axis) plot.

### Example 1

### Synthesis of N,N-Di-(2-aminoethyl)-AmB (2)

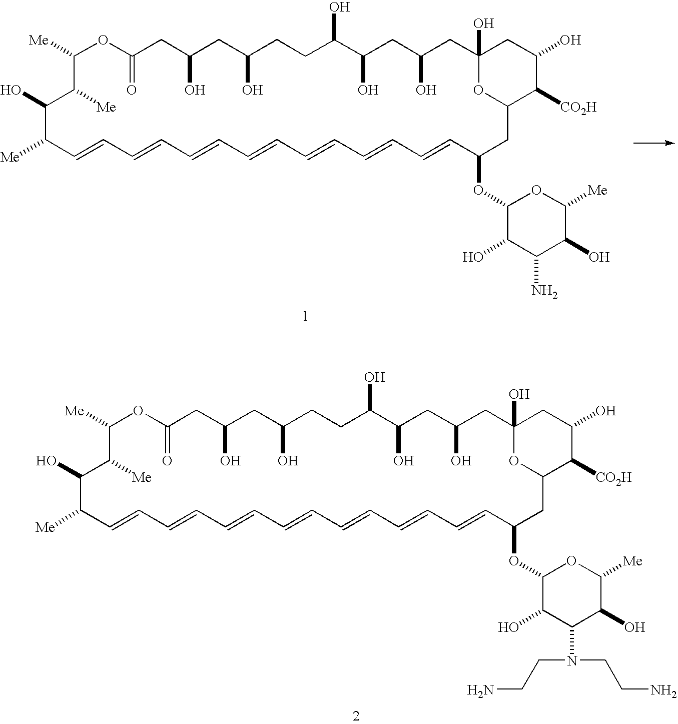

To a solution of N-(9-fluorenylmethoxycarbonyl)-2-aminoacetaldehyde (290 mg, 1.03 mmol) and AmB (1a) (320 mg, 0.340 mmol) in DMF (3.00 mL) was added NaBH₃CN (65.0 mg, 1.03 mmol) followed by a drop of conc. HCl. After 16 h at room temperature, Amberlite IRA-743 (500 mg) was added and the mixture was stirred for an hour. After filtration, the solution was concentrated down and added dropwise to diethyl ether (250 mL). The yellow precipitate was filtered and purified by flash chromatography (40-8-1 CHCl₃-MeOH—H₂O). The isolated yellow solid was dissolved in DMSO (5.00 mL) and piperidine (0.200 mL, 2.10 mmol) was added. After 2 h at room temperature, the solution was added dropwise to diethyl ether (250 mL). The yellow precipitate was filtered and washed with diethyl ether (2×100 mL) providing the desired compound 2 as a yellow solid (70 mg, 74%). ¹H NMR (500 MHz, DMSO-d₆) δ 6.46-6.01 (m, 13H), 5.42 (dd, J=15, 10 Hz, 1H), 5.23 (s (br), 1H), 4.85-4.57 (m, 3H), 4.30-4.24 (m, 3H), 4.06-3.45 (m, 15H), 3.09 (m, 2H), 2.35-2.26 (m, 2H), 2.16 (d, J=6 Hz, 1H), 1.82-1.18 (m, 21H), 1.11 (d, J=6 Hz, 3H), 1.04 (d, J=6 Hz, 3H), 0.92 (d, J=7 Hz, 3H), ¹³C NMR (125 MHz, DMSO-d₆) δ 176.8, 170.5, 136.6, 134.0, 133.6, 133.4, 133.1, 132.7, 132.4, 132.1, 131.8, 131.2, 128.2, 96.8, 77.2, 73.9, 73.5, 73.2, 72.1, 69.1, 68.7, 67.9, 66.1, 66.0, 51.4, 46.5, 44.7, 44.3, 42.5, 42.2, 41.9, 35.1, 30.6, 29.0, 28.3, 18.4, 18.2, 16.8, 12.0; MALDI-TOF calcd for C₅₁H₈₃N₃O₁₇ [M+H⁺]⁺: 1010.5801. Found: 1010.5795.

### N,N-Di-{N-(9-fluorenylmethoxycarbonyl)-3-aminopropyl}-AmB

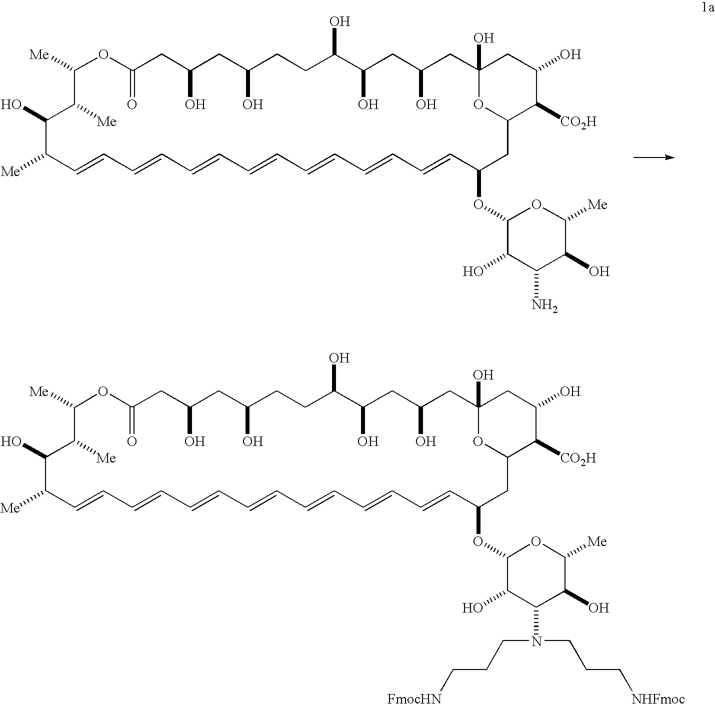

To a solution of N-(9-fluorenylmethoxycarbonyl)-3-aminopropanal (150 mg, 0.510 mmol) and AmB (1a) (157 mg, 0.170 mmol) in DMF (3.00 mL) was added NaBH₃CN (32.0 mg, 0.510 mmol) followed by a drop of conc. HCl. After 16 h at room temperature, Amberlite IRA-743 (300 mg) was added and the mixture was stirred for an hour. After filtration, the solution was concentrated down and added dropwise to diethyl ether (250 mL). The yellow precipitate was filtered and purified by flash chromatography (10-6-1 CHCl₃-MeOH—H₂O) providing the desired compound as a yellow solid (200 mg, 80%). R_{f} 0.70 (10-6-1 CHCl₃-MeOH—H₂O), ¹H NMR (500 MHz, DMSO-d₆) δ 7.87 (d, J=8 Hz, 4H), 7.68 (d, J=8 Hz, 4H), 7.40 (t, J=8 Hz, 4H), 7.31 (t, J=8 Hz, 4H), 6.46-6.06 (m, 12H), 5.96 (dd, J=14, 8 Hz, 1H), 5.84 (s, 1H), 5.44 (dd, J=15, 10 Hz, 1H), 5.33 (s, 1H), 5.21 (s (br), 1H), 4.79-4.60 (m, 4H), 4.43-4.19 (m, 10H), 4.07-3.97 (m, 2H), 3.81 (s, 1H), 3.55-3.41 (m, 3H), 3.31 (s (br), OH), 3.13-3.02 (m, 6H), 2.77 (s, 1H), 2.64 (s, 1H), 2.28 (s, 1H), 2.17 (d, J=6 Hz, 1H), 1.93-1.24 (m, 18H), 1.19 (d, J=6 Hz, 3H), 1.11 (d, J=7 Hz, 3H), 1.04 (d, J=6 Hz, 3H), 0.92 (d, J=7 Hz, 3H), ¹³C NMR (125 MHz, DMSO-d₆) δ 177.6, 170.5, 162.2, 156.0, 143.9, 142.5, 140.6, 139.3, 137.3, 136.7, 133.8, 133.6, 133.4, 133.1, 132.4, 132.3, 132.1, 131.9, 131.8, 131.7, 131.2, 128.8, 127.5, 127.2, 127.0, 125.1, 121.3, 119.9, 101.2, 97.1, 77.1, 77.0, 73.9, 73.5, 69.5, 69.1, 68.8, 67.8, 67.5, 66.1, 65.2, 64.8, 58.2, 48.3, 46.7, 44.8, 44.6, 44.2, 42.0, 41.9, 38.4, 35.7, 35.0, 30.7, 28.9, 28.5, 18.4, 18.2, 16.9, 15.1, 12.0; MALDI-TOF calcd for C₈₇H₁₀₇N₃O₂₁ [M+Na⁺]⁺: 1504.7295. Found: 1504.7289.

### Example 2

### Synthesis of N,N-Di-(3-aminopropyl)-AmB (3)

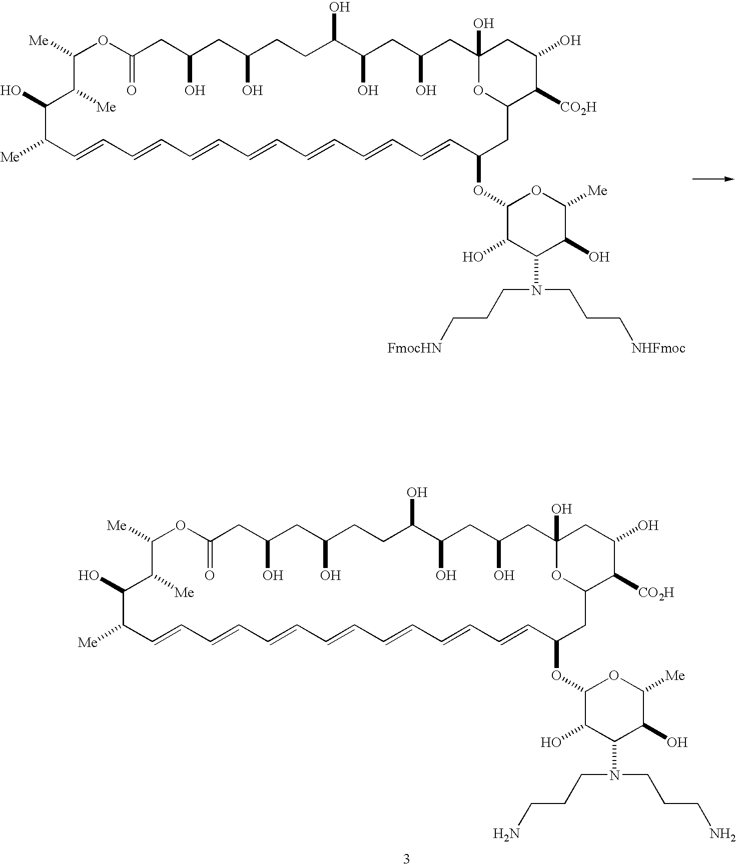

To a solution of N,N-di-{N-(9-fluorenylmethoxycarbonyl)-3-aminopropyl}-AmB (200 mg, 0.135 mmol) in DMSO (3.00 mL) was added piperidine (0.100 mL, 1.06 mmol). After 2 h at room temperature, the solution was added dropwise to diethyl ether (250 mL). The yellow precipitate was filtered and washed with diethyl ether (2×100 mL) providing the desired compound 3 as a yellow solid (135 mg, 95%). R_{f} 0.10 (10-6-1 CHCl₃-MeOH—H₂O), ¹H NMR (500 MHz, DMSO-d₆) δ 6.48-5.97 (m, 13H), 5.42 (dd, J=15, 10 Hz, 1H), 5.23 (s (br), 1H), 4.82-4.66 (m, 3H), 4.46-4.40 (m, 1H), 4.28-4.21 (m, 2H), 4.07-4.02 (m, 1H), 3.94-3.88 (m, 1H), 3.34 (s (br), OH), 3.13-3.07 (m, 3H), 2.82-2.64 (m, 3H), 2.31-2.26 (m, 1H), 2.16 (d, J=6 Hz, 1H), 1.83-1.24 (m, 18H), 1.18 (d, J=6 Hz, 3H), 1.11 (d, J=6 Hz, 3H), 1.04 (d, J=6 Hz, 3H), 0.92 (d, J=7 Hz, 3H), ¹³C NMR (125 MHz, DMSO-d₆) δ 177.5, 170.4, 136.7, 134.0, 133.8, 133.7, 133.1, 132.4, 132.1, 131.9, 131.2, 128.1, 96.5, 77.2, 74.2, 73.9, 73.4, 70.7, 69.1, 68.7, 68.6, 67.8, 66.1, 65.6, 65.5, 65.4, 46.5, 44.7, 44.3, 42.5, 41.9, 38.6, 35.1, 29.0, 18.4, 18.2, 17.1, 16.9, 12.0; MALDI-TOF calcd for C₅₃H₈₇N₃O₁₇ [M+H⁺]⁺: 1038.6114. Found: 1038.6108.

### Example 3

### Synthesis of N,N-Di-(3-hydroxypropyl)-AmB (4)

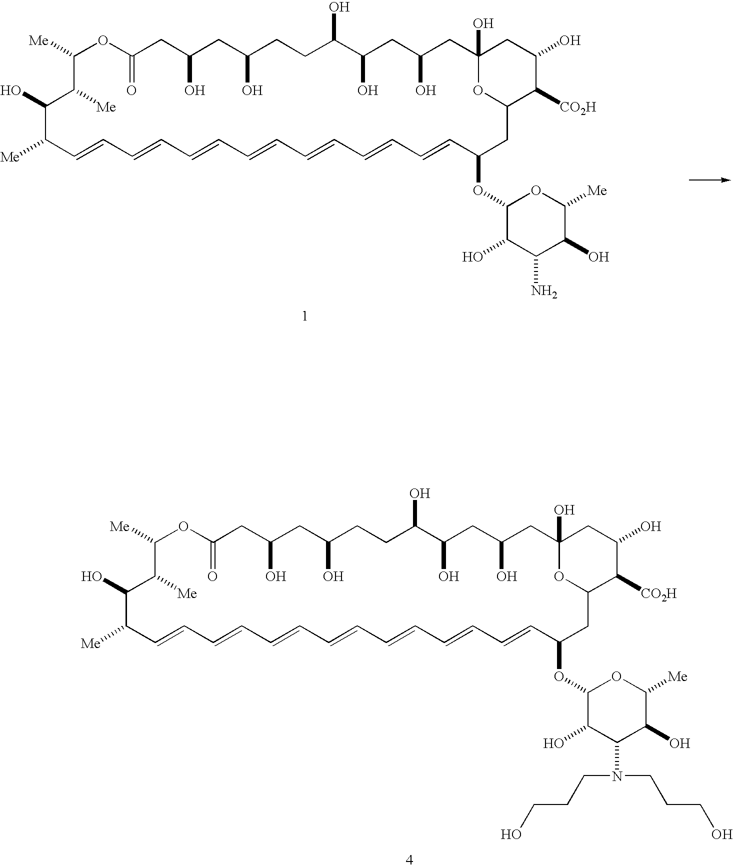

To a solution of 3-(tert-butyldimethylsiloxy)-propanal (300 mg, 1.60 mmol) and AmB (1a) (365 mg, 0.400 mmol) in DMF (3.00 mL) was added NaBH₃CN (100 mg, 1.60 mmol) followed by a drop of conc. HCl. After 16 h at room temperature, Amberlite IRA-743 (400 mg) was added and the mixture was stirred for an hour. After filtration, the solution was concentrated down and added dropwise to diethyl ether (250 mL). The yellow precipitate was filtered and purified by flash chromatography (40-8-1 CHCl₃-MeOH—H₂O). The isolated yellow solid was dissolved in MeOH (5.00 mL) in a plastic bottle. At 0° C., diluted HF.pyridine solution (1.00 mL, prepared from 0.500 mL of the 70% HF.pyridine commercial solution and 0.500 mL of pyridine) was added. After 2 h at room temperature, the solution was concentrated and added dropwise to diethyl ether (250 mL). The yellow precipitate was filtered and washed with diethyl ether (2×100 mL) providing the desired compound 4 as a yellow solid (104 mg, 28%). ¹H NMR (500 MHz, DMSO-d₆) δ 6.47-6.06 (m, 13H), 5.94 (dd, J=15, 9 Hz, 1H), 5.90 (s, 1H), 5.45 (dd, J=15, 10 Hz, 1H), 5.36 (s, 1H), 5.22-5.20 (m, 1H), 4.79-4.76 (m, 2H), 4.72 (s, 1H), 4.61 (s, 1H), 4.45-4.38 (m, 1H), 4.24-4.21 (m, 1H), 4.06-3.92 (m, 2H), 3.60-3.02 (m, OH), 2.31-2.26 (m, 1H), 2.18 (d, J=6 Hz, 1H), 2.00-1.97 (m, 1H), 1.90-1.87 (m, 1H), 1.76-1.72 (m, 2H), 1.60-1.52 (m, 2H), 1.42-1.21 (m, 18H), 1.11 (d, J=6 Hz, 3H), 1.04 (d, J=6 Hz, 3H), 0.92 (d, J=7 Hz, 3H), ¹³C NMR (125 MHz, DMSO-d₆) δ 174.2, 170.5, 136.7, 133.7, 133.5, 133.2, 133.1, 132.5, 132.3, 132.2, 132.0, 131.8, 131.1, 129.0, 97.2, 77.1, 74.6, 73.7, 73.5, 73.1, 69.1, 68.8, 67.6, 66.8, 66.2, 65.3, 65.0, 64.4, 58.7, 56.8, 46.0, 44.7, 44.2, 42.3, 42.0, 36.7, 35.0, 28.9, 18.4, 17.8, 16.9, 12.0; MALDI-TOF calcd for C₅₃H₈₅NO₁₉ [M+H⁺]⁺: 1040.5894. Found: 1040.5789.

### 2,6-Bis-{amino-(9-fluorenylmethoxycarbonyl)}-hexanal

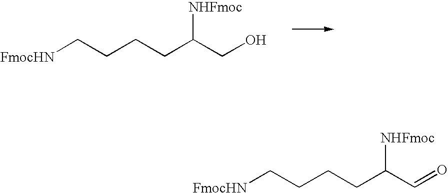

To a solution of 2,6-bis-{amino-(9-fluorenylmethoxycarbonyl)}-hexanol (500 mg, 0.860 mmol) in DCM (50.0 mL) was added Dess-Martin Periodinane (730 mg, 1.72 mmol). After 2 h at room temperature, EtOAc (100 mL) was added to the reaction mixture which was then washed with a saturated aqueous solution Na₂S₂O₅ (2×100 mL). The organic layer was dried over MgSO₄ and the solvent was removed under reduced pressure. The crude aldehyde was purified by flash chromatography (1:1 EtOAc-hexane) providing the desired aldehyde as a white solid (300 mg, 60%). R_{f} 0.4 (1:1 EtOAc-hexane), ¹H NMR (500 MHz, DMSO-d₆) δ 9.44 (s, 1H), 7.88 (d, J=7 Hz, 4H), 7.74 (d, J=8 Hz, 2H), 7.67 (d, J=8 Hz, 2H), 7.41 (t, J=7 Hz, 4H), 7.32 (t, J=7 Hz, 4H), 4.36 (d, J=7 Hz, 2H), 4.30 (d, J=7 Hz, 2H), 4.26-4.18 (m, 2H), 3.87 (m, 1H), 2.97 (q, J=6 Hz, 2H), 1.71 (s (br), 1H), 1.47-1.25 (m, 6H); ¹³C NMR (125 MHz, DMSO-d₆) δ 201.7, 156.4, 156.1, 144.0, 143.8, 140.8, 127.7, 127.6, 127.1, 125.2, 120.2, 65.6, 65.2, 59.7, 46.8, 29.1, 27.3, 22.5; MALDI-TOF calcd for C₃₆H₃₄N₂O₅ [M+Na⁺]⁺: 597.2365. Found: 597.2360.

### Example 4

### Synthesis of N,N-Di-(2,6-diaminohexyl)-AmB (5)

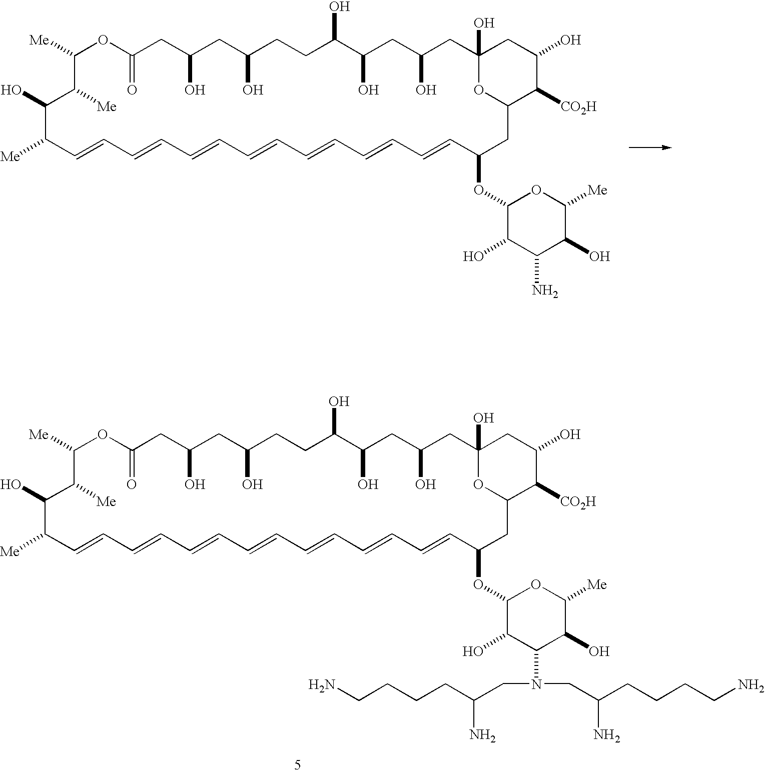

To a solution of AmB (1a) (92 mg, 0.100 mmol) and 2,6-bis-{amino-(9-fluorenylmethoxycarbonyl)}-hexanal (290 mg, 0.500 mmol) in DMF (5.00 mL) was added NaBH₃CN (31.0 mg, 0.500 mmol) followed by a drop of conc. HCl. After 16 h at room temperature, Amberlite IRA-743 (500 mg) was added and the mixture was stirred for an hour. After filtration, the solution was concentrated and added dropwise to diethyl ether (250 mL). The yellow precipitate was filtered and purified by flash chromatography (40-8-1 CHCl₃-MeOH—H₂O). The isolated yellow solid was dissolved in DMSO (2.00 mL) and piperidine (0.100 mL, 1.06 mmol) was added. After 2 h at room temperature, the solution was added dropwise to diethyl ether (250 mL). The yellow precipitate was filtered and washed with diethyl ether (2×100 mL) providing the desired compound 5 as a yellow solid (10 mg, 22%). ¹H NMR (500 MHz, DMSO-d₆) δ 6.40-5.86 (m, 13H), 5.47-5.41 (m, 1H), 5.36 (dd, J=15, 10 Hz, 1H), 5.14 (s (br), 1H), 4.75-4.60 (m, 3H), 4.46-4.44 (m, 1H), 4.27-4.12 (m, 3H), 4.00-3.96 (m, 1H), 3.32 (s (br), OH), 3.06-3.01 (m, 3H), 2.87-2.80 (m, 3H), 2.60-2.50 μm, 1H), 2.24-2.18 (m, 1H), 2.09 (d, J=6 Hz, 1H), 1.73-1.08 (m, 24H), 1.04 (d, J=6 Hz, 3H), 0.96 (d, J=6 Hz, 3H), 0.84 (d, J=7 Hz, 3H), ¹³C NMR (125 MHz, DMSO-d₆) δ 177.4, 170.5, 137.5, 135.2, 134.0, 133.8, 133.6, 133.2, 132.3, 132.1, 131.9, 131.2, 128.8, 96.7, 77.0, 74.2, 73.9, 73.5, 73.2, 70.9, 69.3, 69.1, 68.8, 68.5, 67.8, 66.5, 66.3, 66.1, 65.5, 65.4, 52.6, 40.4, 35.0, 33.2, 29.0, 22.8, 18.4, 18.1, 18.0, 17.0, 16.9, 12.0; MALDI-TOF calcd for C₅₉H₁₀₁N₅O₁₇ [M+H⁺]⁺: 1152.7271. Found: 1152.7265.

### Example 5

### Synthesis of N,N-Di-(2-ethylguanidine)-AmB (6)

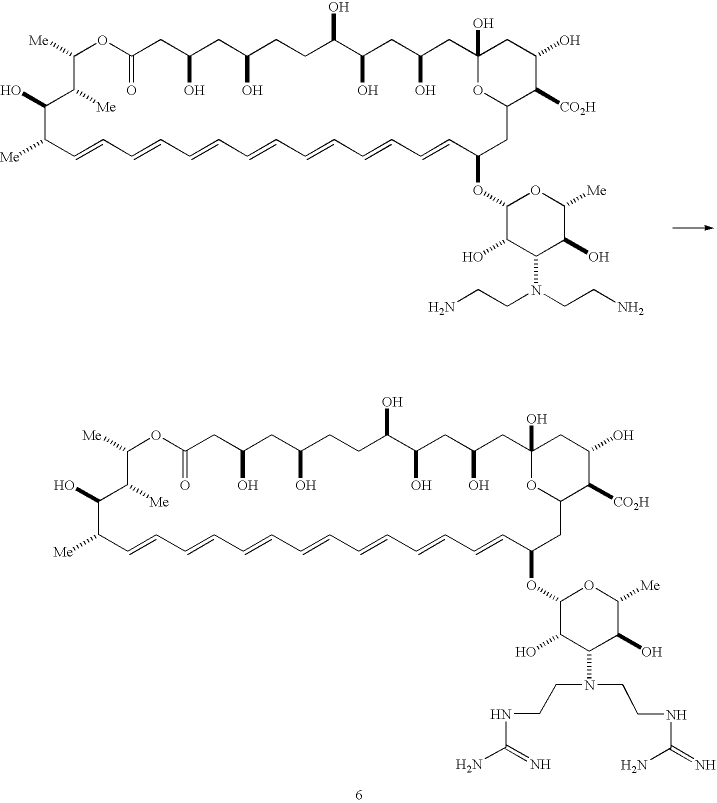

To a solution of N,N-di-(2-aminoethyl)-AmB (100 mg, 0.100 mmol) in DMF (2.00 mL) was added 1H-pyrazole-1-carboxamidine monohydrochloride (36.0 mg, 0.250 mmol) and diisopropylethylamine (0.350 mL, 2.00 mmol). After 48 h at room temperature, the solution was concentrated down and added dropwise to diethyl ether (250 mL). The yellow precipitate was filtered and washed with diethyl ether (2×100 mL) providing the desired compound 6 as a yellow solid (60.0 mg, 55%). ¹H NMR (500 MHz, DMSO-d₆) δ 7.60 (s (br), 1H), 7.42 (s (br), 2H), 6.52-6.02 (m, 13H), 5.43 (dd, J=15, 10 Hz, 1H), 5.23 (s (br), 1H), 4.80-4.72 (m, 3H), 4.64 (s, 1H), 4.45-4.22 (m, 3H), 4.08-3.98 (m, 2H), 3.53 (m, 18H, OH), 3.15-3.08 (m, 1H), 2.32-2.26 (m, 1H), 2.17 (d, J=6 Hz, 1H), 1.83-1.23 (m, 18H), 1.19 (d, J=6 Hz, 3H), 1.11 (d, J=6 Hz, 3H), 1.04 (d, J=6 Hz, 3H), 0.92 (d, J=7 Hz, 3H), ¹³C NMR (125 MHz, DMSO-d₆) δ177.6, 170.5, 157.1, 142.1, 136.7, 133.8, 133.6, 133.4, 133.1, 132.4, 132.1, 131.8, 131.2, 128.5, 97.6, 96.9, 77.1, 74.1, 73.9, 73.5, 69.1, 68.7, 68.4, 67.9, 66.1, 65.5, 65.1, 64.8, 63.6, 50.0, 45.9, 44.7, 42.7, 41.9, 41.0, 40.4, 35.7, 30.7, 29.0, 24.9, 23.1, 18.6, 18.4, 18.3, 18.0, 16.8, 12.0; MALDI-TOF calcd for C₅₃H₈₈N₇O₁₇ [M+H⁺]⁺: 1094.6237. Found: 1094.6231.

### Example 6

### Synthesis of 2,6-Diamino-N-[6-oxo-6-(piperazin-1-yl)-hexyl]-hexanamide-AmB (7)

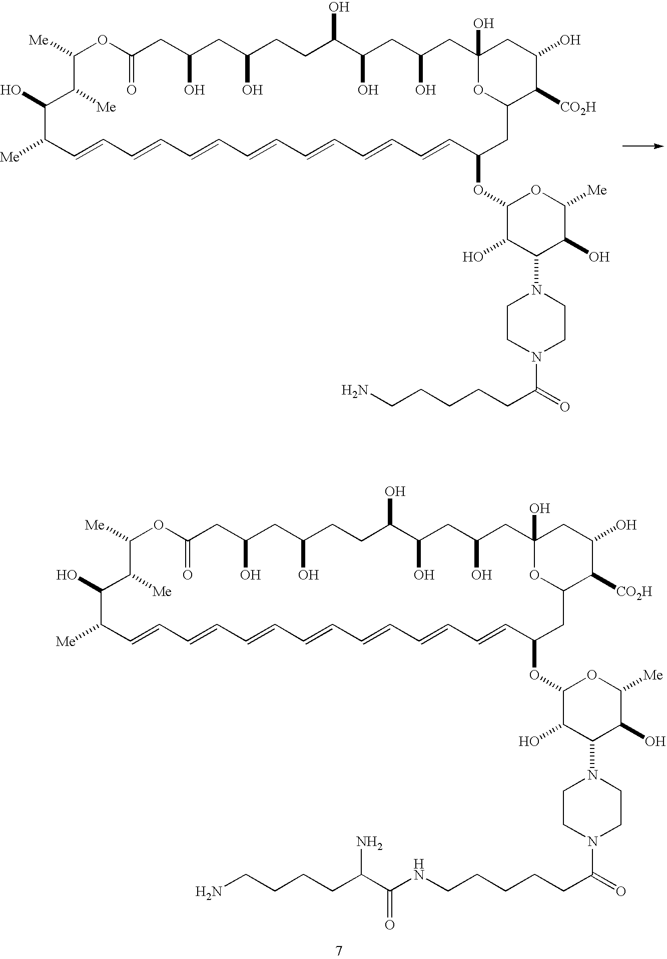

To a solution of 6-amino-1-hexanoyl-(piperazinyl)-AmB (Zumbuehl, A. et al., Angew. Chem. Int. Ed. 2004, 43, 5181) (110 mg, 0.100 mmol) in DMF (2.00 mL) was added 2,6-di-{N-(9-fluorenylmethoxycarbonyl)-lysine hydroxysuccinimide ester (Ben-Ami, S. and L. Yehuda, Cancer Treatment Reports 1981, 65, 277-281) (380 mg, 0.500 mmol) and diisopropylethylamine (0.100 mL, 0.500 mmol). After 18 h at room temperature, the solution was concentrated down and added dropwise to diethyl ether (250 mL). The yellow precipitate was filtered and purified by flash chromatography (40-8-1 CHCl₃-MeOH—H₂O). The isolated yellow solid was dissolved in DMSO (3.00 mL) and piperidine (0.100 mL, 1.00 mmol) was added. After 2 h at room temperature, the solution was added dropwise to diethyl ether (250 mL). The yellow precipitate was filtered and washed with diethyl ether (2×100 mL) providing the desired compound 7 as a yellow solid (10 mg, 27%). ¹H NMR (500 MHz, DMSO-d₆) δ 7.78 (s (br), 1H), 6.46-5.95 (m, 13H), 5.51 (dd, J=15, 10 Hz, 1H), 5.44 (dd, J=15, 10 Hz, 1H), 5.20 (s (br), 1H), 5.13-5.11 (m, 1H), 4.79-4.70 (m, 3H), 4.48 (s, 1H), 4.29-4.14 (m, 4H), 4.06 (s, 2H), 3.90-3.79 (m, 2H), 3.38 (s, OH), 3.14-3.04 (m, 2H), 2.72-2.58 (m, 2H), 2.38-2.21 (m, 3H), 2.16 (d, J=6 Hz, 1H), 1.74-1.24 (m, 21H), 1.17 (d, J=6 Hz, 3H), 1.11 (d, J=6 Hz, 3H), 1.04 (d, J=6 Hz, 3H), 0.91 (d, J=7 Hz, 3H), ¹³C NMR (125 MHz, DMSO-d₆) δ 174.9, 173.8, 170.4, 170.2, 137.5, 136.6, 133.9, 133.7, 133.6, 133.5, 133.2, 132.7, 132.3, 132.0, 131.8, 131.4, 128.3, 96.7, 77.1, 73.8, 73.5, 72.2, 69.3, 69.2, 68.8, 67.7, 67.6, 67.5, 67.3, 66.2, 66.1, 65.9, 65.5, 64.8, 54.6, 44.6, 44.3, 42.3, 42.0, 41.9, 35.0, 34.9, 30.7, 28.9, 26.1, 26.0, 25.6, 24.5, 22.3, 18.4, 18.2, 17.1, 16.9, 12.1, 12.0; MALDI-TOF calcd for C₆₃H₁₀₃N₅O₁₉ [M+Na⁺]⁺: 1256.7145. Found: 1256.7140.

### Example 7

### Synthesis of 2-Amino-6-guanidine-N-[6-oxo-6-(piperazin-1-yl)-hexyl]-hexanamide-AmB (8)

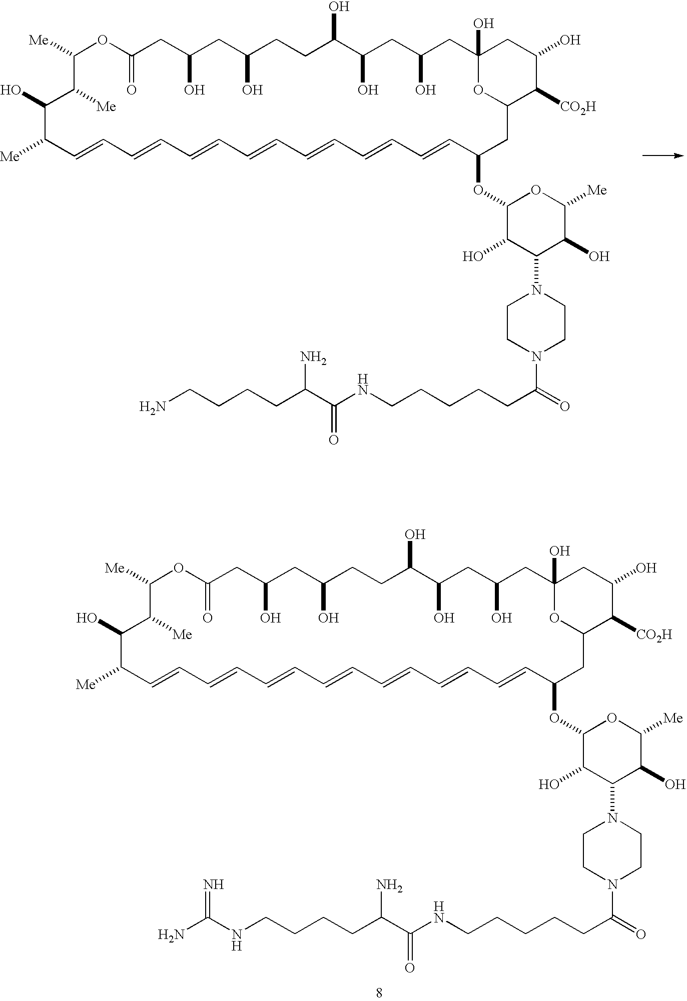

To a solution of 2,6-diamino-N-[6-oxo-6-(piperazin-1-yl)-hexyl]-hexanamide-AmB (50.0 mg, 0.0400 mmol) in DMF (2.00 mL) was added 1H-pyrazole-1-carboxamidine monohydrochloride (8.00 mg, 0.0500 mmol) and diisopropylethylamine (0.0170 mL, 0.100 mmol). After 18 h at room temperature, the solution was concentrated down and added dropwise to diethyl ether (250 mL). The yellow precipitate was filtered and washed with diethyl ether (2×100 mL) providing the desired compound 8 as a yellow solid (10.0 mg, 20%). ¹H NMR (500 MHz, DMSO-d₆) δ 7.60 (s (br), 1H), 6.49-5.91 (m, 14H), 5.44 (dd, J=15, 10 Hz, 1H), 5.37 (s (br), 1H), 5.21 (s (br), 1H), 4.81-4.64 (m, 3H), 4.44-4.31 (m, 1H), 4.28-4.16 (m, 2H), 4.07-4.04 (m, 1H), 4.00-3.96 (m, 1H), 3.83-3.79 (m, 2H), 3.34 (s, OH), 3.09-3.06 (m, 2H), 2.32-2.24 μm, 3H), 2.16 (d, J=6 Hz, 1H), 1.85-1.22 (m, 18H), 1.17 (d, J=6 Hz, 3H), 1.11 (d, J=6 Hz, 3H), 1.04 (d, J=6 Hz, 3H), 0.91 (d, J=7 Hz, 3H), ¹³C NMR (125 MHz, DMSO-d₆) δ 174.7, 173.5, 170.5, 170.2, 157.1, 136.7, 136.1, 133.8, 133.6, 133.4, 133.1, 132.7, 132.4, 132.1, 131.8, 131.5, 131.1, 128.6, 96.9, 77.0, 74.4, 73.8, 73.6, 73.5, 69.2, 69.1, 68.7, 67.7, 67.4, 67.2, 66.1, 65.5, 65.4, 65.3, 64.9, 53.4, 46.1, 46.0, 44.7, 44.3, 43.6, 42.0, 35.0, 28.9, 28.5, 28.3, 25.1, 24.8, 24.5, 22.2, 22.0, 18.4, 18.1, 16.9, 13.8, 12.0; MALDI-TOF calcd for C₆₄H₁₀₅N₇O₁₉ [M+Na⁺]⁺: 1298.7363. Found: 1298.7358.

### Example 8

### Synthesis of N,N-Di-(3-aminopropyl)-AmB Methyl Ester (9)

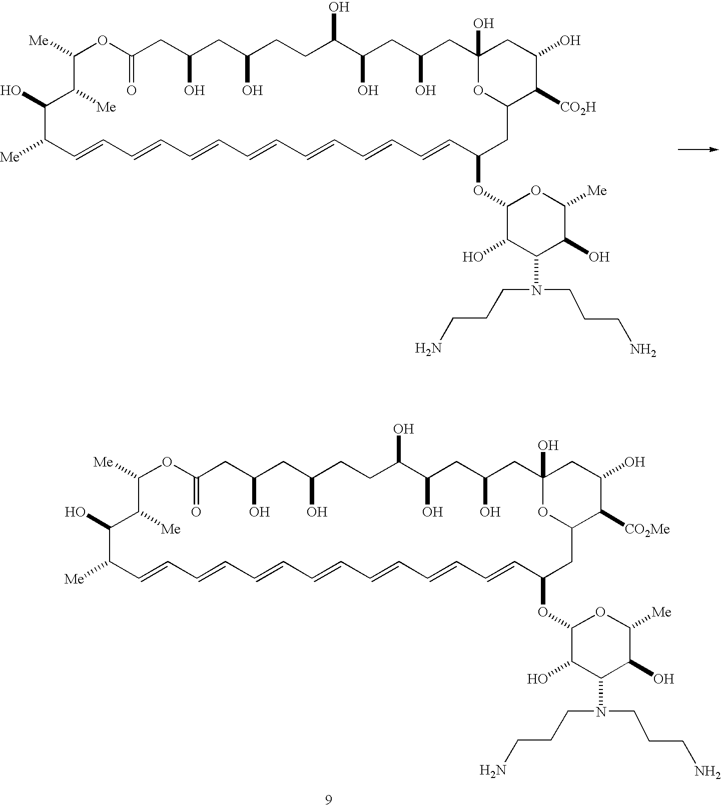

To a solution of N,N-di-(3-aminopropyl)-AmB (150 mg, 0.140 mmol) in MeOH (10.0 mL) was added a 2M solution of trimethylsilyldiazomethane in diethyl ether (0.700 mL, 14.0 mmol). After 3 h at room temperature, the solution was concentrated down to a volume of 1.00 mL and then added dropwise to diethyl ether (250 mL). The yellow precipitate was filtered and washed with diethyl ether (2×100 mL) providing the desired compound 9 as a yellow solid (90.0 mg, 59%). ¹H NMR (500 MHz, DMSO-d₆) δ 6.45-5.93 (m, 13H), 5.44 (dd, J=15, 10 Hz, 1H), 5.34 (s (br), 1H), 5.22 (s (br), 1H), 4.92-4.69 (m, 3H), 4.35-4.18 (m, 3H), 4.06-4.02 (m, 1H), 3.81-3.76 (m, 1H), 3.63 (s, 3H), 3.34 (s (br), OH), 3.10-3.04 (m, 3H), 2.82-2.63 (m, 3H), 2.39-2.24 (m, 1H), 2.17 (d, J=6 Hz, 1H), 1.89-1.28 (m, 18H), 1.18 (d, J=6 Hz, 3H), 1.11 (d, J=6 Hz, 3H), 1.04 (d, J=6 Hz, 3H), 0.91 (d, J=7 Hz, 3H), ¹³C NMR (125 MHz, DMSO-d₆) δ 173.0, 170.4, 136.7, 134.1, 133.8, 133.1, 132.5, 132.1, 131.7, 131.2, 129.2, 97.2, 96.8, 80.2, 76.2, 75.6, 74.0, 73.1, 69.0, 68.1, 66.2, 65.3, 64.8, 64.0, 52.1, 51.1, 45.6, 44.8, 44.0, 42.1, 41.9, 28.8, 18.4, 18.2, 17.1, 16.9, 12.0; MALDI-TOF calcd for C₅₄H₈₉N₃O₁₇ [M+H⁺]⁺: 1052.6270. Found: 1052.6265.

### Example 9

### Synthesis of 16-(N′-(2-Aminoethyl)-carboxamide)-N,N-di-(3-aminopropyl)-AmB (10)

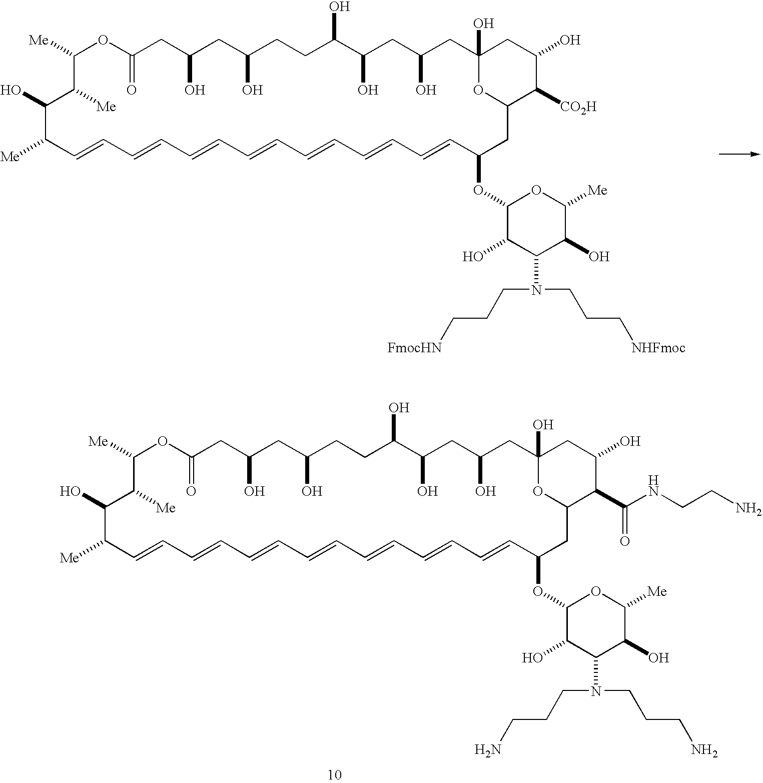

To a solution of N,N-di-{N-(9-fluorenylmethoxycarbonyl)-3-aminopropyl}-AmB (170 mg, 0.115 mmol) in DMF (3.00 mL) was added N-Fmoc-ethylenediamine (49.0 mg, 0.172 mmol), 1-hydroxybenzotriazole (19.0 mg, 0.138 mmol), (benzotriazol-1-yloxy)-tripyrrolidinophosphonium hexafluorophosphate (60.0 mg, 0.115 mmol) and diisopropylethylamine (40.00 L, 0.230 mmol). After 36 h at room temperature, the solution was concentrated down and a flash chromatography (40-8-1 CHCl₃-MeOH—H₂O) provided the protected derivative of compound 10. The yellow solid was dissolved in DMSO (3.00 mL) and was added piperidine (0.200 mL, 2.10 mmol). After 2 h at room temperature, the solution was added dropwise to diethyl ether (250 mL). The yellow precipitate was filtered and washed with diethyl ether (2×100 mL) providing the desired compound 10 as a yellow solid (44.0 mg, 35%). ¹H NMR (500 MHz, DMSO-d₆) δ 6.48-6.06 (m, 13H), 5.96-5.91 (m, 1H), 5.43 (dd, J=15, 10 Hz, 1H), 5.22 (s (br), 1H), 4.89-4.65 (m, 3H), 4.46-4.40 (m, 1H), 4.30-4.18 (m, 2H), 4.09-4.00 (m, 1H), 3.94-3.88 (m, 1H), 3.88-3.44 (s (br), OH), 3.14-3.00 (m, 7H), 2.88-2.64 (m, 3H), 2.31-2.26 (m, 1H), 2.17 (d, J=6 Hz, 1H), 1.62-1.23 (m, 18H), 1.19 (d, J=6 Hz, 3H), 1.11 (d, J=6 Hz, 3H), 1.04 (d, J=6 Hz, 3H), 0.92 (d, J=7 Hz, 3H), ¹³C NMR (125 MHz, DMSO-d₆) δ 171.8, 170.5, 136.7, 133.8, 133.6, 133.3, 133.1, 132.4, 132.1, 131.8, 131.2, 128.5, 97.0, 77.1, 74.1, 73.8, 73.4, 69.0, 68.7, 67.8, 67.7, 67.6, 67.3, 66.1, 65.5, 64.8, 64.4, 54.2, 46.4, 45.8, 45.7, 44.7, 44.0, 42.4, 41.9, 38.0, 35.0, 29.0, 25.8, 18.4, 18.2, 16.8, 12.0; MALDI-TOF calcd for C₅₅H₉₃N₅O₁₆ [M+H⁺]⁺: 1080.6696. Found: 1080.61690.

### Example 10

### Synthesis of 16-(N′-(3-Dimethylaminopropyl)carboxamide)-N,N-di-(3-aminopropyl)-AmB (11)

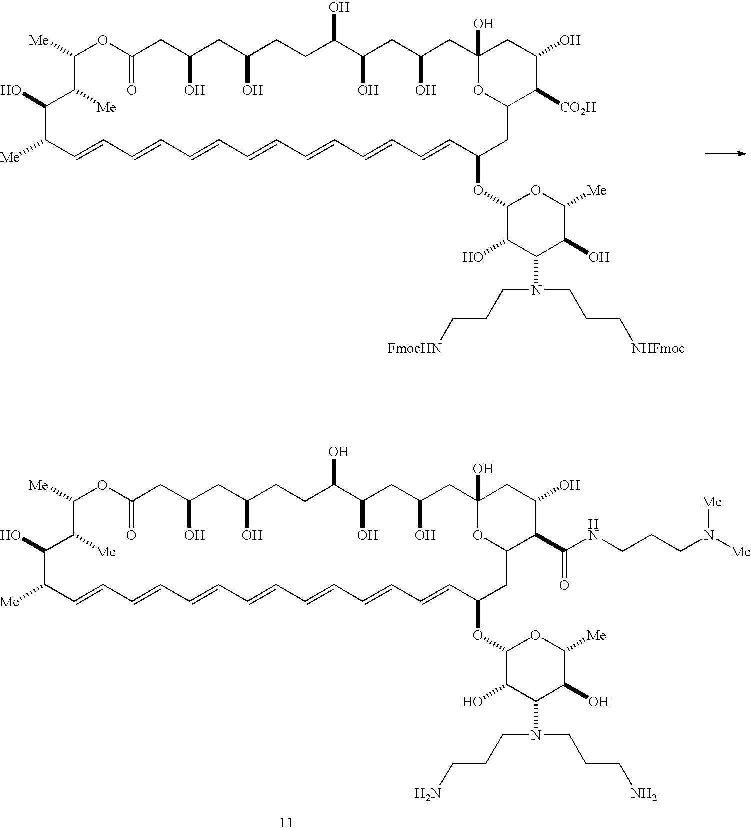

To a solution of N,N-di-{N-(9-fluorenylmethoxycarbonyl)-3-aminopropyl}-AmB (100 mg, 0.068 mmol) in DMF (2.00 mL) was added 3-dimethylaminopropylamine (17.0 mg, 0.136 mmol), 1-hydroxybenzotriazole (11.0 mg, 0.082 mmol), (benzotriazol-1-yloxy)-tripyrrolidinophosphonium hexafluorophosphate (35.0 mg, 0.068 mmol) and diisopropylethylamine (20.0 μL, 0.102 mmol). After 24 h at room temperature, the solution was concentrated down and the residue was added dropwise to diethyl ether (250 mL). The yellow precipitate was filtered and washed with diethyl ether (2×100 mL). To the yellow solid in DMSO (3.00 mL) was added piperidine (0.200 mL, 2.10 mmol). After 2 h at room temperature, the solution was added dropwise to diethyl ether (250 mL). The yellow precipitate was filtered and washed with diethyl ether (2×100 mL) providing the desired compound 11 as a yellow solid (36.0 mg, 47%). MALDI-TOF calcd for C₅₈H₉₉N₅O₁₆ [M+H⁺]⁺: 1122.4306. Found: 1122.7160.

### Example 11

### Synthesis of 16-(N′-(1-Methylpiperazinyl)carboxamide)-N,N-di-(3-aminopropyl)-AmB (12)

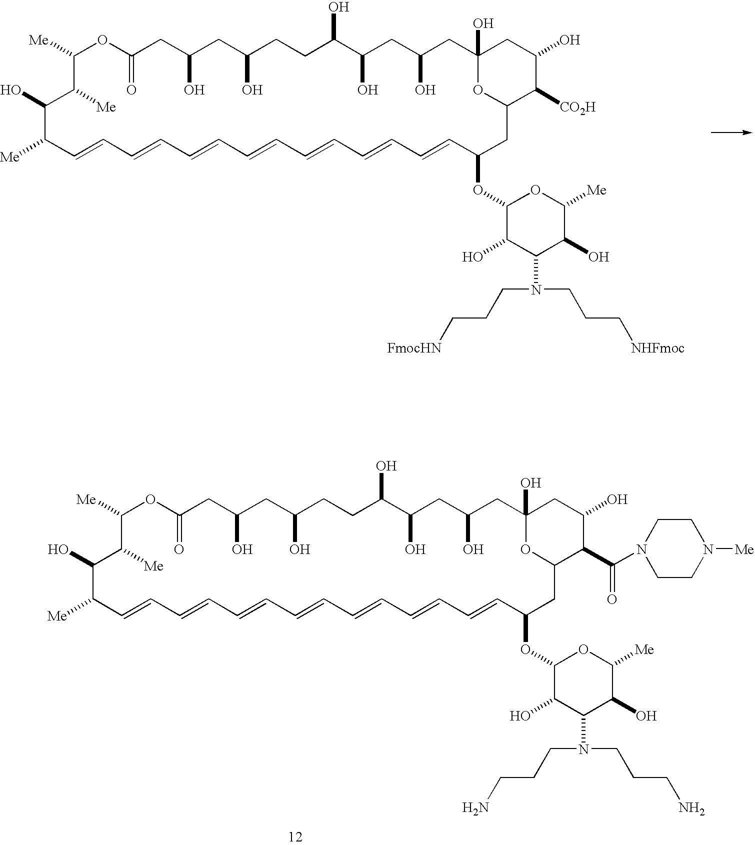

To a solution of N,N-di-{N-(9-fluorenylmethoxycarbonyl)-3-aminopropyl}-AmB (100 mg, 0.068 mmol) in DMF (2.00 mL) was added 1-methylpiperazine (15.0 mg, 0.136 mmol), 1-hydroxybenzotriazole (11.0 mg, 0.082 mmol), (benzotriazol-1-yloxy)tripyrrolidinophosphonium hexafluorophosphate (35.0 mg, 0.068 mmol) and diisopropylethylamine (20.0 μL, 0.102 mmol). After 24 h at room temperature, the solution was concentrated down and the residue was added dropwise to diethyl ether (250 mL). The yellow precipitate was filtered and washed with diethyl ether (2×100 mL). To the yellow solid in DMSO (3.00 mL) was added piperidine (0.200 mL, 2.10 mmol). After 2 h at room temperature, the solution was added dropwise to diethyl ether (250 mL). The yellow precipitate was filtered and washed with diethyl ether (2×100 mL) providing the desired compound 12 as a yellow solid (40.0 mg, 38%). MALDI-TOF calcd for C₅₈H₉₇N₅O₁₆ [M+Na⁺]⁺: 1142.7016. Found: 1142.6823.

### Example 12

### Synthesis of 16-(N′-(4-(3-aminopropyl)-morpholine)carboxamide)-N,N-di-(3-aminopropyl)-AmB (13)

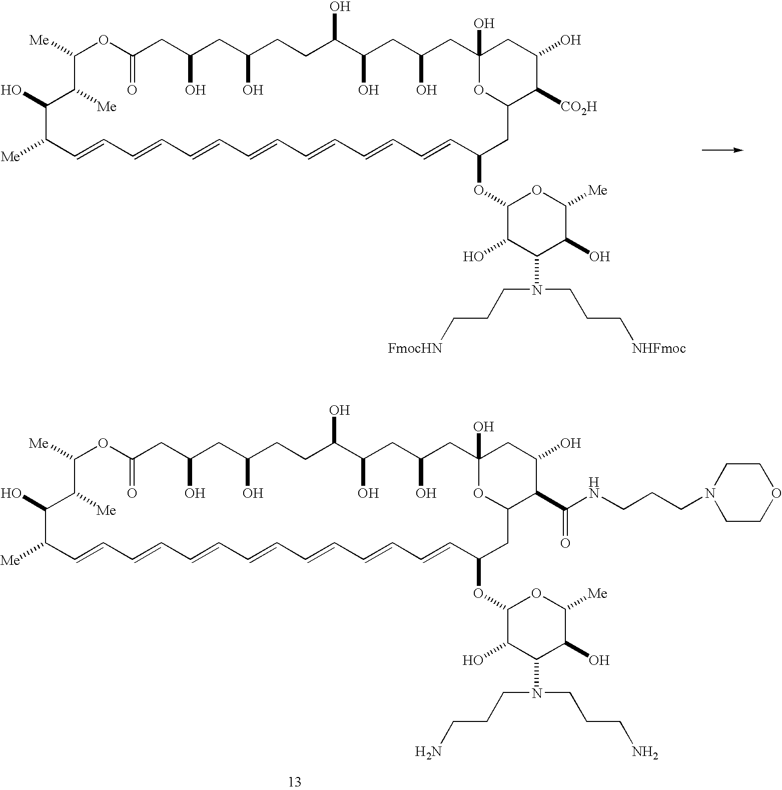

To a solution of N,N-di-{N-(9-fluorenylmethoxycarbonyl)-3-aminopropyl}-AmB (100 mg, 0.068 mmol) in DMF (2.00 mL) was added 4-(3-aminopropyl)-morpholine (20.0 mg, 0.136 mmol), 1-hydroxybenzotriazole (11.0 mg, 0.082 mmol), (benzotriazol-1-yloxy)-tripyrrolidinophosphonium hexafluorophosphate (35.0 mg, 0.068 mmol) and diisopropylethylamine (20.00 L, 0.102 mmol). After 24 h at room temperature, the solution was concentrated down and the residue was added dropwise to diethyl ether (250 mL). The yellow precipitate was filtered and washed with diethyl ether (2×100 mL). To the yellow solid in DMSO (3.00 mL) was added piperidine (0.200 mL, 2.10 mmol). After 2 h at room temperature, the solution was added dropwise to diethyl ether (250 mL). The yellow precipitate was filtered and washed with diethyl ether (2×100 mL) providing the desired compound 13 as a yellow solid (39.0 mg, 49%). MALDI-TOF calcd for C₆₀H₁₀₁N₅O₁₇ [M+H⁺]⁺: 1164.4672. Found: 1164.7265.

### Example 13

### Synthesis of N-(3-aminopropyl)-N-(2-aminoethyl)-AmB (14)

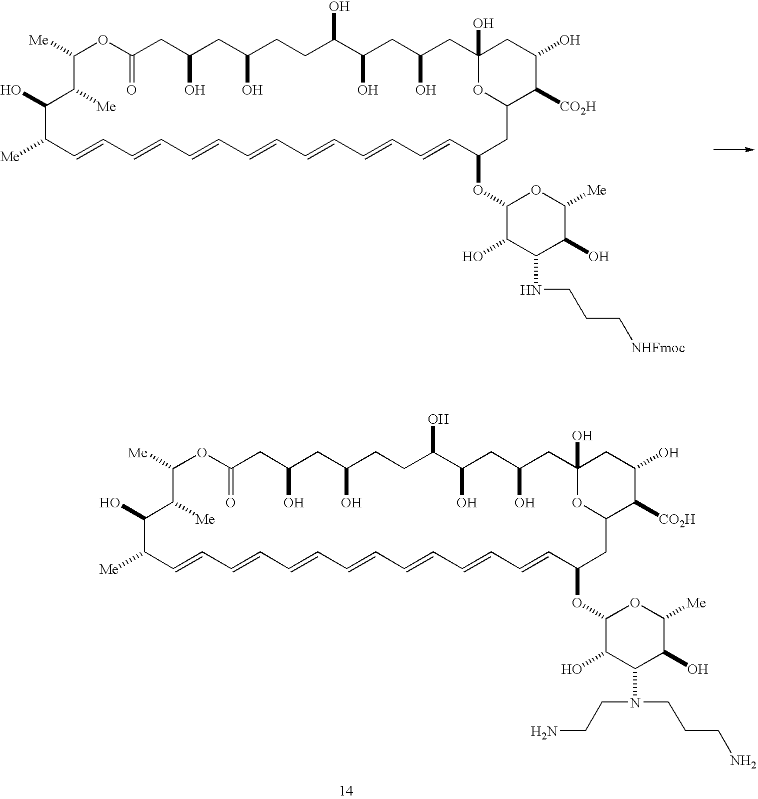

To a solution of N-(9-fluorenylmethoxycarbonyl)-2-aminoacetaldehyde (Gang, W. et al., Org. Lett. 2003, 5, 737) (113 mg, 0.400 mmol) in DMF (3.00 mL) and MeOH (3.00 mL) was added to the previously isolated N-(9-fluorenylmethoxycarbonyl)-3-aminopropyl-AmB (240 mg, 0.200 mmol). After 3 h, NaBH₃CN (40.0 mg, 0.600 mmol) was added to the mixture. After 16 h at room temperature, Amberlite IRA-743 (500 mg) was added and the mixture was stirred for an hour. After filtration, the solution was concentrated and added dropwise to diethyl ether (250 mL). The yellow precipitate was filtered and purified by flash chromatography (40-8-1 CHCl₃-MeOH—H₂O). The isolated yellow solid was dissolved in DMSO (5.00 mL) and piperidine (0.200 mL, 2.10 mmol) was added. After 2 h at room temperature, the solution was added dropwise to diethyl ether (250 mL). The yellow precipitate was filtered and washed with diethyl ether (2×100 mL) providing the desired compound as a yellow solid.

### Synthesis of N-(9-fluorenylmethoxycarbonyl)-3-aminopropyl-AmB

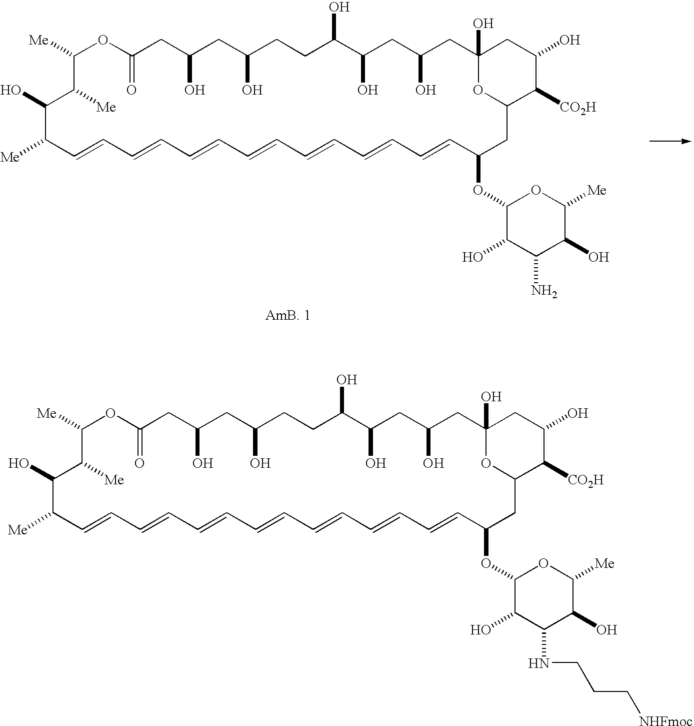

To a solution of N-(9-fluorenylmethoxycarbonyl)-3-aminopropanal (More, J. D. and Finney, N. S. Org. Lett. 2002, 4, 3001) (59.0 mg, 0.200 mmol) in DMF (3.00 mL) and MeOH (3.00 mL) was added AmB (1) (216 mg, 0.200 mmol). After 3 h, NaBH₃CN (40.0 mg, 0.600 mmol) was added to the mixture. After 16 h at room temperature, Amberlite IRA-743 (500 mg) was added and the mixture was stirred for an hour. After filtration, the solution was concentrated down and added dropwise to diethyl ether (250 mL). The yellow precipitate was filtered and purified by flash chromatography (40-8-1 CHCl₃-MeOH—H₂O) providing the desired compound as a yellow solid.

### Example 14

### Synthesis of N,N-Di-(methyl-4-butanoate)-AmB (15)

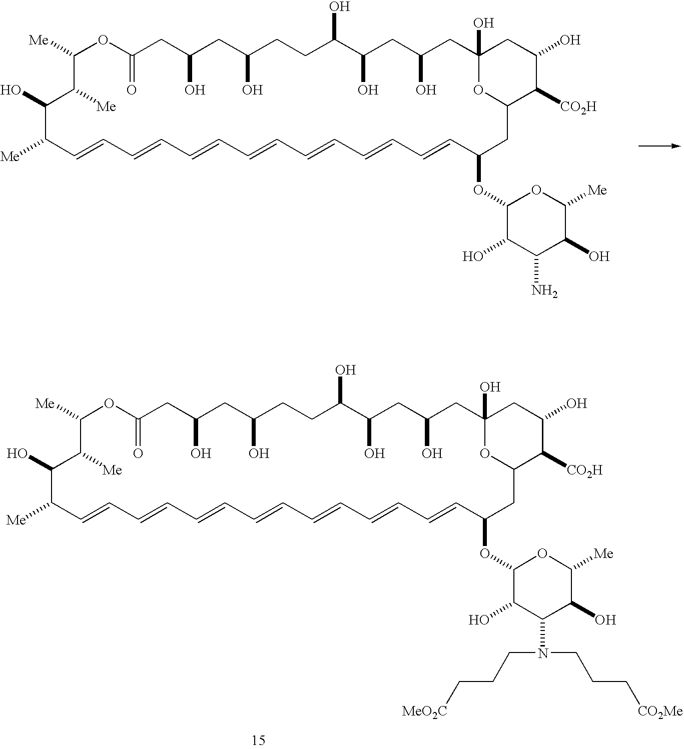

To a solution of methyl-4-oxobutanoate (520 mg, 4.50 mmol) and AmB (1) (820 mg, 0.890 mmol) in DMF (5.00 mL) was added NaBH₃CN (280 mg, 4.50 mmol) followed by a drop of conc. HCl. After 18 h at room temperature, Amberlite IRA-743 (800 mg) was added and the mixture was stirred for an hour. After filtration, the solution was concentrated down and added dropwise to diethyl ether (250 mL). The yellow precipitate was filtered and purified by flash chromatography (40-8-1 CHCl₃-MeOH—H₂O) providing the desired compound 15 as a yellow solid (450 mg, 45%). R_{f} 0.30 (40-8-1 CHCl₃-MeOH—H₂O), ¹H NMR (500 MHz, DMSO-d₆) δ 6.46-6.06 (m, 13H), 5.95 (dd, J=15, 9 Hz, 1H), 5.82 (s, 1H), 5.45 (dd, J=15, 10 Hz, 1H), 5.32 (s, 1H), 5.21-5.19 (m, 1H), 4.80-4.74 (m, 2H), 4.62 (s, 1H), 4.43-4.36 (m, 1H), 4.32 (s, 1H), 4.25-4.17 (m, 2H), 4.06-3.96 (m, 2H), 3.75 (s, 1H), 3.58 (s, 6H), 3.55-3.35 (m, OH), 3.11-3.08 (m, 1H), 2.78-2.72 (m, 1H), 2.64-2.59 (m, 1H), 2.31 (t, J=7 Hz, 4H), 2.17 (d, J=6 Hz, 1H), 1.98-1.23 (m, 21H), 1.18 (d, J=6 Hz, 3H), 1.11 (d, J=6 Hz, 3H), 1.04 (d, J=6 Hz, 3H), 0.92 (d, J=7 Hz, 3H), ¹³C NMR (125 MHz, DMSO-d₆) δ 173.6, 173.2, 170.5, 136.9, 136.7, 133.8, 133.6, 133.5, 133.1, 132.4, 132.3, 132.1, 132.0, 131.8, 131.6, 131.1, 128.7, 97.4, 97.1, 80.3, 77.1, 76.3, 75.7, 74.4, 73.8, 73.7, 73.5, 69.4, 69.2, 68.9, 67.9, 67.6, 66.2, 65.4, 63.9, 56.9, 51.0, 50.2, 46.2, 44.6, 44.2, 42.3, 42.0, 35.0, 31.0, 29.0, 24.0, 18.4, 18.2, 16.9, 12.0; MALDI-TOF calcd for C₅₅H₈₅NO₂₁ [M+H⁺]⁺: 1124.6005. Found: 1124.5600.

### Example 15

### Synthesis of N,N-Di-(4-butanoic acid)-AmB (16)

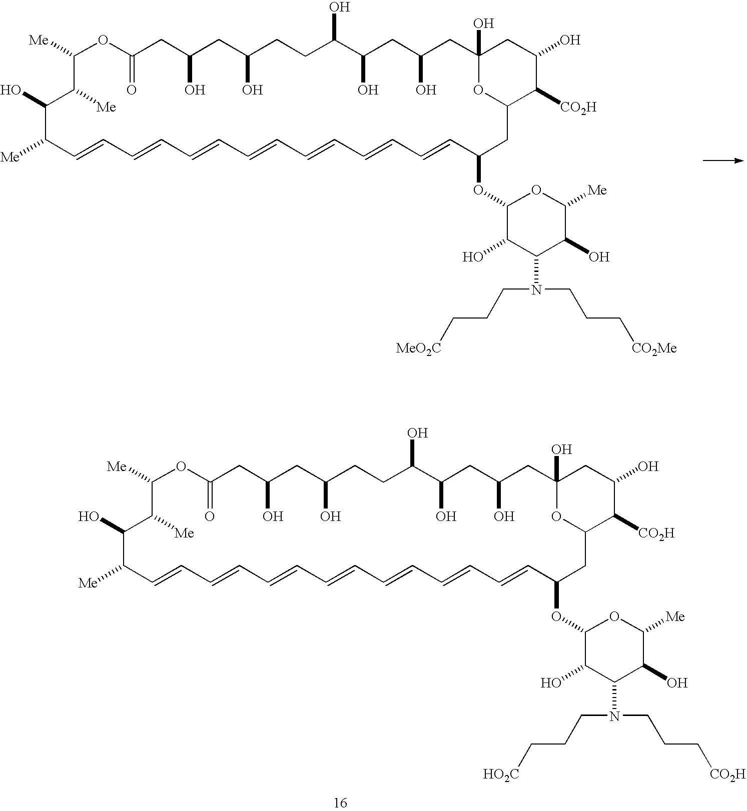

To a solution of N,N-di-(methyl-4-butanoate)-AmB (100 mg, 0.089 mmol) in THF (5.00 mL) and H₂O (3.00 mL) was added aqueous LiOH solution (2.00 mL, 1.00 M) at 0° C. After 1 h, the reaction mixture was acidified to pH 5 with dilute aqueous hydrochloric acid solution. The solution was concentrated and added dropwise to diethyl ether (250 mL). The yellow-brown precipitate was filtered providing the desired compound 16 (42.0 mg, 43%). ¹H NMR (500 MHz, DMSO-d₆) δ 12.22 (s (br), 2H), 6.60-5.85 (m, 15H), 5.55-5.34 (s, 2H), 5.25-5.04 (m, 2H), 5.02-4.99 (m, 1H), 4.95-4.90 (m, 1H), 4.85-4.82 (m, 2H), 4.64-4.57 (m, 1H), 4.39 (s, 1H), 4.15-4.05 (m, 1H), 3.68 (m, OH), 3.33-3.12 (m, 1H), 2.37-2.30 (m, 2H), 2.17 (d, J=6 Hz, 1H), 2.00-1.90 (m, 1H), 1.71-1.28 (m, 21H), 1.18 (dd, J 10, 6 Hz, 3H), 1.11 (d, J=6 Hz, 3H), 1.04 (d, J=6 Hz, 3H), 0.91 (d, J=7 Hz, 3H), ¹³C NMR (125 MHz, DMSO-d₆) δ 173.7, 173.5, 170.4, 136.9, 136.7, 133.8, 133.6, 133.5, 133.1, 132.4, 132.3, 132.1, 132.0, 131.8, 131.6, 131.1, 128.7, 97.8, 97.1, 93.9, 93.3, 80.7, 77.1, 76.4, 75.9, 75.2, 73.9, 73.5, 73.3, 72.4, 68.9, 68.5, 67.7, 66.4, 66.0, 64.8, 61.6, 51.0, 50.2, 46.2, 44.6, 42.3, 42.0, 35.0, 31.2, 18.5, 18.4, 16.9, 12.0; MALDI-TOF calcd for C₅₅H₈₅NO₂₁ [M−H]⁻: 1094.5536. Found: 1094.5541.

### Example 16

### Synthesis of N,N-Di-(3-aminopropyl)-Nystatin (17)

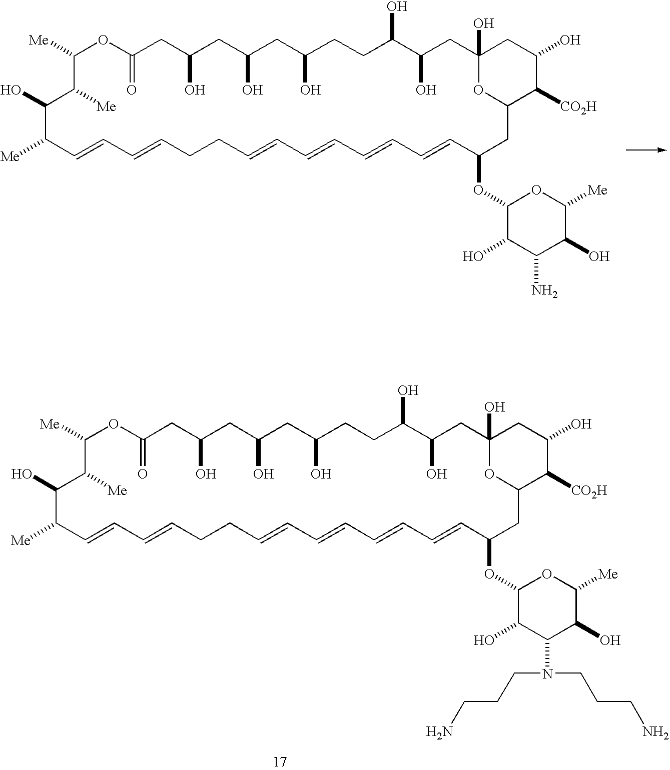

To a solution of N-(9-fluorenylmethoxycarbonyl)-3-aminopropanal (100 mg, 0.355 mmol) and Nystatin (1b) (115 mg, 0.118 mmol) in DMF (2.00 mL) was added NaBH₃CN (22.0 mg, 0.355 mmol) followed by a drop of conc. HCl. After 16 h at room temperature, Amberlite IRA-743 (500 mg) was added and the mixture was stirred for an hour. After filtration, the solution was concentrated down and added dropwise to diethyl ether (250 mL). The yellow precipitate was filtered and purified by flash chromatography (10-6-1 CHCl₃-MeOH—H₂O) providing the protected compound 17 as a yellow solid (150 mg, 87%). To a solution of N,N-di-(N-(9-fluorenylmethoxycarbonyl)-3-aminopropyl)-Nystatin (150 mg, 0.135 mmol) in DMSO (2.00 mL) was added piperidine (0.100 mL, 1.06 mmol). After 2 h at room temperature, the solution was added dropwise to diethyl ether (250 mL). The yellow precipitate was filtered and washed with diethyl ether (2×100 mL) providing the desired compound 17 as a yellow solid (87 mg, 71%). ¹H NMR (500 MHz, DMSO-d₆) δ 6.33-6.07 (m, 8H), 5.97-5.93 (m, 4H), 5.71-5.65 (m, 1H), 5.60-5.41 (m, 2H), 5.078-503 (m, 1H), 4.70-4.65 (m, 1H), 4.44 (s (br), 1H), 4.28 (s (br), 1H), 4.20-4.16 (m, 1H), 4.04-3.93 (m, 2H), 3.88-3.77 (m, 2H), 3.67-3.41 (m, 3H), 3.17-3.13 (m, 3H), 2.95-2.69 (m, 3H), 2.36-2.09 (m, 2H), 1.85-1.36 (m, 20H), 1.20 (d, J=6 Hz, 3H), 1.10 (d, J=6 Hz, 3H), 0.97 (dd, J=17, 6 Hz, 3H), 0.89 (dd, J=5, 7 Hz, 3H), ¹³C NMR (125 MHz, DMSO-d₆) δ 177.4, 170.5, 134.8, 133.6, 132.7, 132.6, 132.1, 131.6, 131.5, 131.4, 131.0, 130.6, 129.6, 129.1, 96.8, 76.2, 74.2, 73.3, 73.1, 71.1, 70.6, 70.5, 70.4, 70.3, 69.9, 69.4, 69.0, 68.0, 65.5, 64.8, 44.4, 42.3, 40.3, 18.2, 17.9, 17.0, 16.5, 12.0; MALDI-TOF calcd for C₅₃H₈₉N₃O₁₇ [M+H⁺]⁺: 1040.6270. Found: 1040.6265.

### Example 17

### Synthesis of N,N-Di-(3-aminopropyl)-Pimaricin (18)

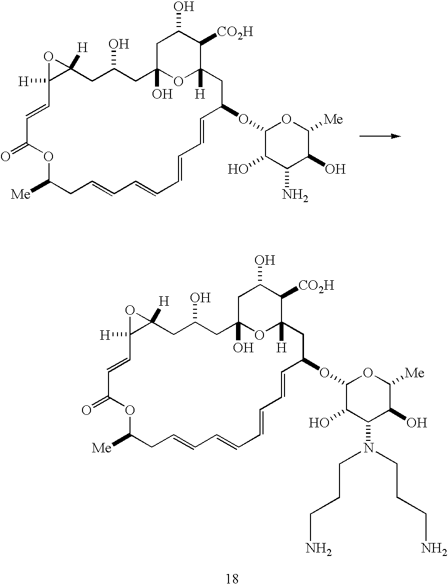

To a solution of N-(9-fluorenylmethoxycarbonyl)-3-aminopropanal (65.0 mg, 0.230 mmol) and Pimaricin (1c) (50 mg, 0.075 mmol) in DMF (2.00 mL) was added NaBH₃CN (14.0 mg, 0.230 mmol) followed by a drop of conc. HCl. After 16 h at room temperature, Amberlite IRA-743 (300 mg) was added and the mixture was stirred for an hour. After filtration, the solution was concentrated down and added dropwise to diethyl ether (250 mL). The yellow precipitate was filtered and purified by flash chromatography (40-8-1 CHCl₃-MeOH—H₂O). To the isolated yellow solid in DMSO (2.00 mL) was added piperidine (0.100 mL, 1.06 mmol). After 2 h at room temperature, the solution was added dropwise to diethyl ether (250 mL)). The yellow precipitate was filtered and washed with diethyl ether (2×100 mL) providing the desired compound 18 as a yellow solid (13 mg, 22%), ¹H NMR (500 MHz, DMSO-d₆) δ 6.53 (dd, J=14, 11 Hz, 1H), 6.28-6.02 (m, 7H), 5.89 (dd, J=15, 10 Hz, 1H), 5.63-5.57 (m, 1H), 5.38 (s (br), 1H), 4.67-4.61 (m, 1H), 4.42 (s (br), 1H), 4.26-4.14 (m, 2H), 3.99-3.91 (m, 2H), 3.51-3.30 (s (br), 12H), 3.21 (dd, J=8, 1 Hz, 1H), 3.13-3.10 (m, 1H), 2.82-2.73 (m, 6H), 2.40-2.36 (m, 1H), 2.32-2.16 (m, 1H), 1.98 (d, J=13 Hz, 1H), 1.79-1.45 (m, 6H), 1.37-1.32 (m, 1H), 1.26 (d, J=6 Hz, 3H), 1.18 (d, J=6 Hz, 3H), 1.12-1.04 (m, 1H), ¹³C NMR (125 MHz, DMSO-d₆) δ 176.9, 164.4, 144.6, 135.5, 133.6, 131.9, 131.2, 131.0, 128.8, 128.6, 127.7, 127.2, 124.7, 121.3, 119.9, 96.6, 76.5, 74.3, 70.8, 69.6, 68.6, 66.2, 65.3, 62.2, 58.2, 57.8, 53.4, 47.6, 46.3, 44.2, 40.3, 37.6, 26.9, 20.2, 18.2; MALDI-TOF calcd for C₃₉H₆₁N₃O₁₃ [M+H⁺]⁺: 780.4283. Found: 780.4277.

### Example 18

### Determination of MIC Values of AmB Derivatives in Saccharomyces cerevisiae Wild Type BY4741

MIC values of various polyene macrolides of the invention in Saccharomyces cerevisiae wild type BY4741, a derivative of S288C, (with AmB as a reference) were determined as described hereinabove. Table 1 shows that all of the derivatives showed a lower minimal inhibitory concentration (MIC) required to completely inhibit growth of Saccharomyces cerevisiae compared to AmB. Superior results were in particular achieved with the Diamine-AmB conjugate 3, which was 15 times more active than AmB with a MIC value of 0.02 μM. Compounds 9 and 10, which represent an ester and amide derivative of compound 3, were also 3 and 7.5 times more active than AmB, respectively. This high potency was in particular unprecedented since ester and amide derivatives have previously been reported to be typically less active than native AmB (1a) (Chéron, M. et al Biochem. Pharmacol. 1988, 37, 827; Slisz, M. et al, J. Antibiot. 2004, 10, 669; Carmody, M. et al J. Biol. Chem. 2005, 280, 34420).

TABLE 1
MIC values of selected polyene macrolide derivatives of
formula IIIa

| | | | | IIIa |
|---|---|---|---|---|
| 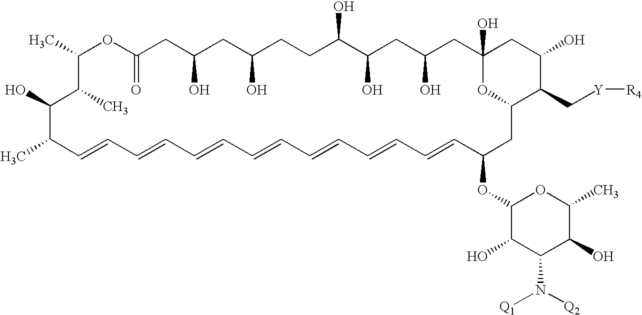 | | | | |
| | | | | MIC |
| # | Q₁ | Q₂ | —Y—R₄ | (μM) |
| 1a, AmB | —H | —H | —OH | 0.3 |
| 2 | —CH₂—CH₂—NH₂ | —CH₂—CH₂—NH₂ | —OH | 0.25 |
| 3 | —CH₂—CH₂—CH₂—NH₂ | —CH₂—CH₂—CH₂—NH₂ | —OH | 0.020 |
| 4 | —CH₂—CH₂—CH₂—OH | —CH₂—CH₂—CH₂—OH | —OH | 0.50 |
| 5 | —CH₂—CH(NH₂)—(CH₂)₄—NH₂ | —CH₂—CH(NH₂)—(CH₂)₄—NH₂ | —OH | 0.080 |
| 6 | 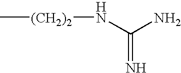 | 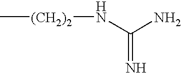 | —OH | 0.10 |
| 7 | 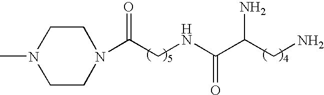 | | —OH | 0.10 |
| 8 | 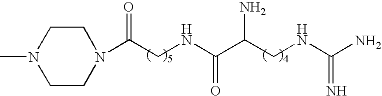 | | —OH | 0.10 |
| 9 | —CH₂—CH₂—CH₂—NH₂ | —CH₂—CH₂—CH₂—NH₂ | —OCH₃ | 0.10 |
| 10 | —CH₂—CH₂—CH₂—NH₂ | —CH₂—CH₂—CH₂—NH₂ | —NH—CH₂—CH₂—NH₂ | 0.040 |
| 11 | —CH₂—CH₂—CH₂—NH₂ | —CH₂—CH₂—CH₂—NH₂ | —NH—CH₂—CH₂—NH(CH₃)₂ | 0.40 |
| 12 | —CH₂—CH₂—CH₂—NH₂ | —CH₂—CH₂—CH₂—NH₂ | 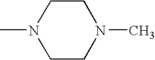 | 0.20 |

### Example 18

### Determination of MIC Values of Selected Polyene Macrolides in Various Candida Strains

MIC values of the polyene macrolides 3, 9 and 10 (Diamine-AmB and its ester and diamide derivative, respectively) in various Candida strains including an AmB-resistant strain C. albicans (DSY1764, with the genotype erg3AΔ::hisG/erg3BΔ::hisG erg11Δ::hisG/erg11Δ::hisG-URA3-hisG) were determined. Table 2 shows that the polyene macrolide 3 was more active then AmB over a wide range of different yeast strains both with clinical isolates and with mutant strains. In the case of the AmB resistant Candida albicans strain DSY 1764 the polyene macrolide 3 displayed a dramatic increase in activity over AmB with MIC=1.0 μM. Polyene macrolides 9 and 10 also exhibited significant inhibitory activity against this resistant strain with each having a MIC value of 4.0 μM.

TABLE 2
MIC values of polyene macrolides 3, 9 and 10 in various Candida strains

| | | | MIC (μM) | MIC (μM) | MIC (μM) | MIC (μM) |
|---|---|---|---|---|---|---|
| | | MIC (μM) | AmB- | AmB- | AmB- | AmB- |
| Candida | MIC (μM) | AmB- | diamine | diamine | diamine | diamine |
| strain | AmB, 1a | diamine 3 | ester 9 | amide 10 | amide 11 | amide 12 |
| DSY-294 | 0.40 | 0.20 | 0.25 | 1.0 | 0.50 | 0.30 |
| C. albican | | | | | | |
| CI | | | | | | |
| DSY-296 | 0.40 | 0.10 | 0.10 | 0.75 | 0.30 | 0.30 |
| C. albican | | | | | | |
| CI | | | | | | |
| DSY-562 | 0.50 | 0.20 | 0.25 | 0.50 | 0.30 | 0.30 |
| C. glabrata | | | | | | |
| CI | | | | | | |
| DSY-565 | 0.50 | 0.20 | 0.25 | 0.50 | 0.30 | 0.20 |
| C. glabrata | | | | | | |
| CI | | | | | | |
| CAF2-1 | 0.30 | 0.10 | 0.50 | 2.0 | 0.50 | 0.30 |
| C. albican | | | | | | |
| wt | | | | | | |
| DSY-654 | 0.50 | 0.10 | 0.25 | 2.0 | 0.50 | 0.40 |
| C. albican | | | | | | |
| mutant | | | | | | |
| cdr1/2 | | | | | | |
| DSY-1751 | 0.30 | 0.10 | 0.50 | 2.0 | 1.0 | 0.40 |
| C. albican | | | | | | |
| mutant | | | | | | |
| erg3 | | | | | | |
| DSY-1764 | 50 | 1.0 | 4.0 | 4.0 | 1.0 | 1.0 |
| C. albican | | | | | | |
| mutant | | | | | | |
| erg3/11 | | | | | | |

### Example 19

### Toxicity (EH₅₀)

The toxicity of AmB (1a) and the polyene macrolides 3, 9 and 10 towards human erythrocytes was examined using the above described hemolysis assay (Table 3). Table 3 shows that AmB derivative 3 (EH₅₀=10 μM) was 2.5 times less toxic than AmB (1a) (EH₅₀=4.0 μM), while 9 displayed even less toxicity (EH₅₀=50 μM), a result consistent with previous observations involving the less active AmB methyl ester (Keim, G. R. et al Science 1973, 179, 584-585). Significantly, 10 was not only a highly active antifungal agent but also displayed much lower toxicity (EH₅₀=30 μM).

TABLE 3
EH₅₀ values

| Compounds | MIC [μM] | EH₅₀ [μM] |
|---|---|---|
| AmB 1 | 0.30 | 4.0 |
| Diamine 3 | 0.02 | 10 |
| Diamine Ester 9 | 0.10 | 50 |
| Diamine Amide 10 | 0.04 | 30 |

### Example 20

### K+ Efflux Measurements

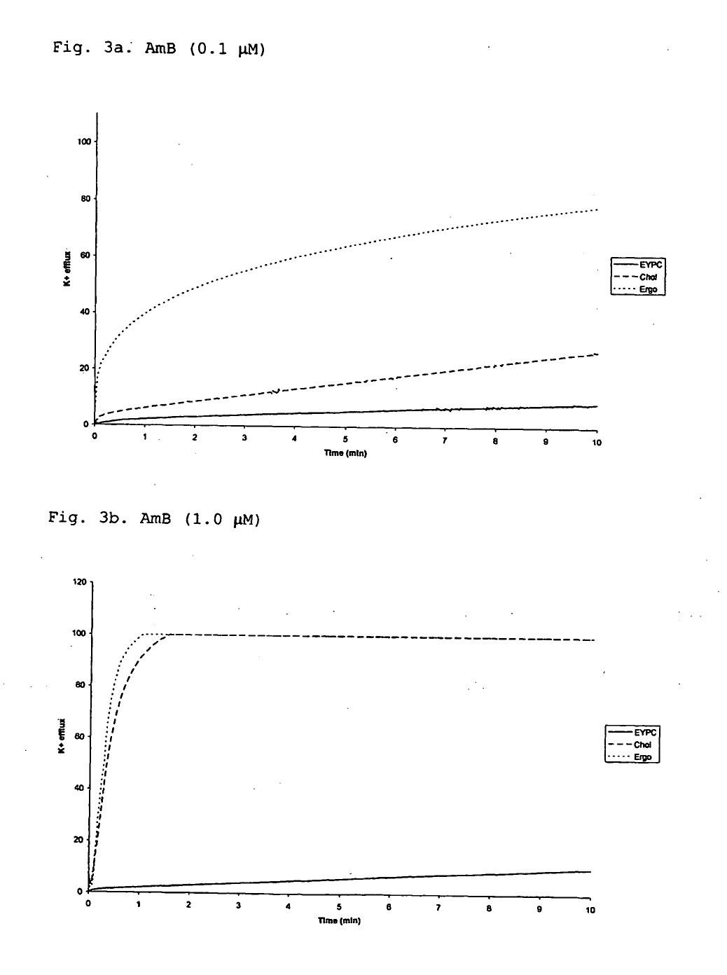

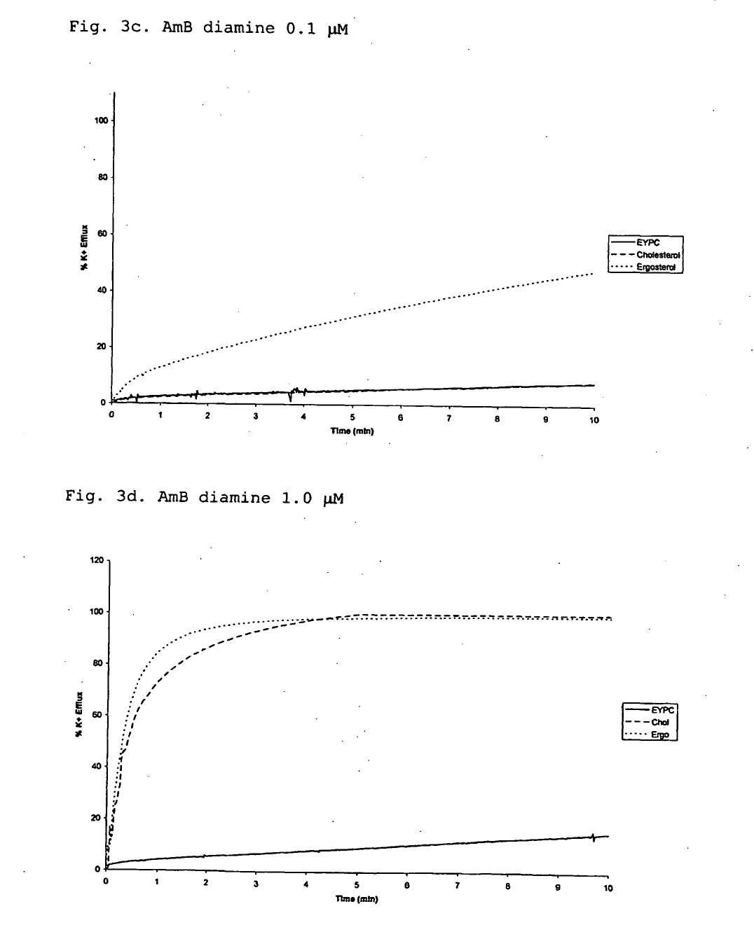

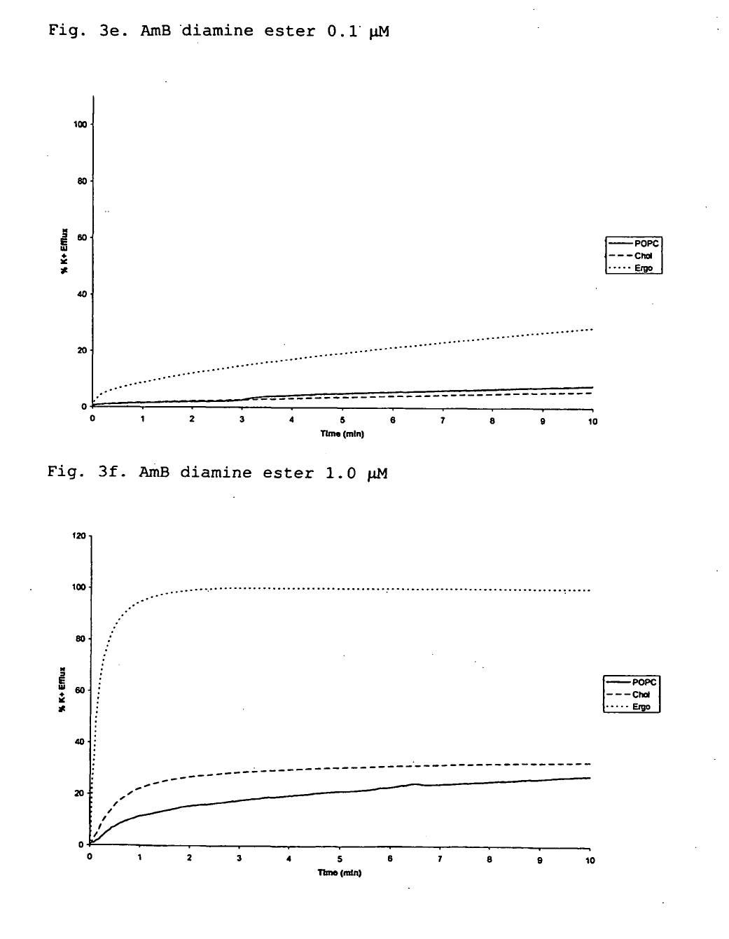

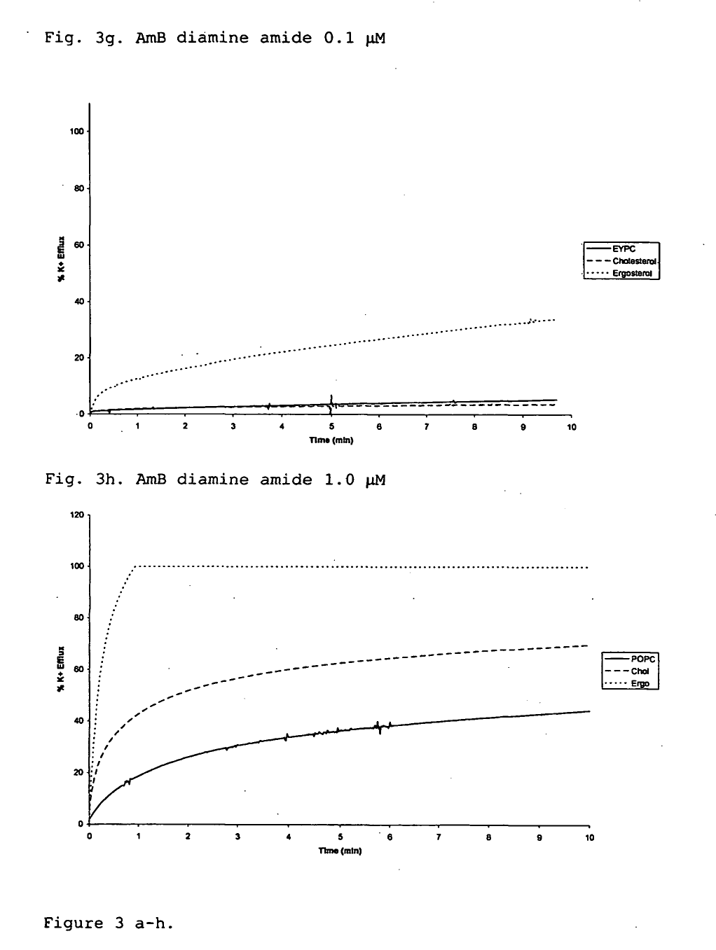

As described hereinbefore differential interaction between ergosterol found in fungal membranes and cholesterol in mammalian cells is considered the basis for the selectivity of AmB (1a) as an antifungal agent. The preferential stabilization of ion channel formation in the membrane by ergosterol ultimately results in electrolyte leakage which can be conveniently measured by K⁺ efflux from sterol containing vesicles. To assess the interaction of the polyene macrolide derivatives of the invention with sterols, derivatives 3, 9 and 10 were examined in their ability to lead to K⁺ efflux from large unilamellar vesicles (LUV) prepared from POPC (1-palmitoyl-2-oleoyl-sn-glycero-3-phosphocholine) admixed with sterols, mimicking conditions in natural biomembranes (FIG. 3). At high concentration (FIG. 3b,d,f,h), all compounds rapidly induce complete K⁺ efflux from vesicles containing ergosterol (dotted line). Derivatives 9 and 10 caused significantly reduced efflux with cholesterol-containing vesicles (broken line), indicating improved selectivity over AmB (1a). In contrast to AmB (1a), at lower concentration (FIG. 3,a,c,e,f), the K⁺ efflux with derivatives 3, 9 and 10 was observed exclusively with the ergosterol containing vesicles (dotted line) when compared against cholesterol containing vesicles (broken line), which is indistinguishable from background (solid line). This differentiation between ergosterol and cholesterol is consistent with the observation of reduced hemotoxicity of 3, 9 and 10.

### Example 21

### Solubility

The solubility of the polyene macrolides of the invention in water was tested following the above described protocol. Clearly, all conjugates were more soluble in water then AmB. In particular, the polyene macrolide 10 (Diamine-Amide) was four times more soluble with up to 40 μg/mL of water.

TABLE 4
Solubility measurements

| | Solubility |
|---|---|
| Compounds | in water [μg/ml] |
| AmB 1 | 10 |
| Diamine 3 | 12 |
| Diamine-Ester 9 | 22 |
| Diamine-Amide 10 | 40 |

### Example 22

### Determination of MIC Values of Nystatin and Pimaricin Derivatives in Saccharomyces cerevisiae wt BY4741

Based on the remarkable performance observed with the Diamine-AmB conjugate similar modifications of the aminosugar moiety were made on other polyene macrolides, such as for example Nystatin (1b) and Pimaricin (1c). The antifungal activity of these nystatin and pimaricin conjugates bearing two aminopropyl side chains was measured using Saccharomyces cerevisiae wild type BY4741. Table 5 shows that the diamine conjugates 17 and 18 were significantly more active than the native compounds 1b and 1c indicating that activity enhancement by attaching the diamine moiety could be generalized to other polyene macrolides.

TABLE 5
MIC values of Nystatin- and Pimaricin derivatives

| Compounds | MIC (μM) |
|---|---|
| 1b (Nystatin) | 3.0 |
| 17 | 0.05 |
| 1c (Pimaricin) | 4.0 |
| 18 | 0.30 |

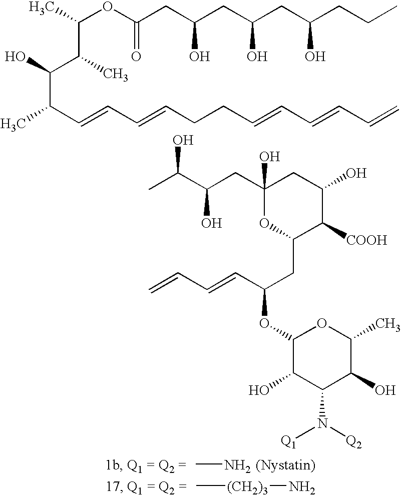

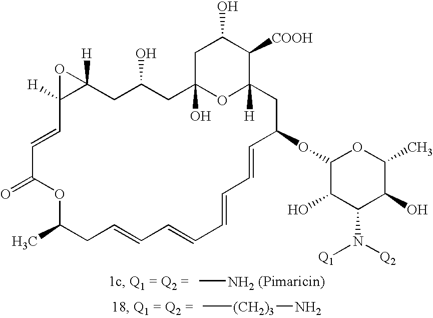

1. Polyene macrolide derivatives according to formula (I): 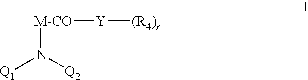
or a pharmaceutically acceptable salt thereof,
wherein:
M represents a polyene macrolide backbone;
Q₁ and Q₂ represent
(i) a group of formula —(R₁)—(X₁)ₘ and —(R₂)—(X₂)ₙ, respectively, wherein
X₁, X₂ represent independently of each other a basic group, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
R₁, R₂ represent independently of each other an unsubstituted or substituted hydrocarbon group, selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; or
(ii) taken together with the adjacent nitrogen atom to which they are attached, a nitrogen-containing heterocyclic group substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

2. Polyene macrolide according to claim 1, wherein the polyene macrolide backbone is selected from amphotericin B, nystatin, candidin, candicidin, aureofacin, levorin, mycoheptin, partricin, perimycin, pimaricin, polyfungin, rimocidin and trichomycin.

3. Polyene macrolide according to claim 1, wherein X₁, X₂ and X₃ represent independently of each other a basic group selected from —NHR₅, —OH, —C(═NH)—NHR₅, —NH—C(═NH)—NHR₅, —N₃, —COR₅, —COOR₅, and —CONHR₅, wherein R₅ represents hydrogen or C(1-10)alkyl.

4. Polyene macrolide according to claim 1, wherein Y is O, N or NH.

5. Polyene macrolide according to claim 1, wherein R₁, R₂ and R₃ represent linear or branched C(1-10)alkyl, C(4-10)cycloalkyl or C(4-10)heterocycloalkyl, which are unsubstituted or substituted by —NH₂, —OH, —COOR₅, —CONHR₅, or —CN, and in which one or more —CH₂-groups of the alkyl groups are optionally replaced by —O—, —CO—, —COO—, —NR₅—, —NR₅CO—, —C(═NH)—NH—, —CH═CH—, wherein R₅ independently represents hydrogen or alkyl.

6. Polyene macrolide according to claim 1, wherein R₁ and R₂ are the same.

7. Polyene macrolide according to claim 1, wherein X₁ and X₂ are the same.

8. Polyene macrolide according to claim 1 having the structure of formula II: 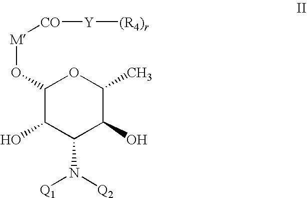
or a pharmaceutically acceptable salt thereof,
wherein:
M′ represents the macrocyclic lactone ring of a polyene macrolide backbone;
Q₁ and Q₂ represent
(i) a group of formula —(R₁)—(X₁)ₚ and —(R₂)—(X₂)_{q}, respectively, wherein
X₁, X₂ represent independently of each other a basic group, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
R₁, R₂ represent independently of each other an unsubstituted or substituted hydrocarbon group, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; or
(ii) taken together with the adjacent nitrogen atom to which they are attached, a nitrogen-containing heterocyclic group substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

9. Polyene macrolide according to claim 1 having the structure of formulae III a-c: 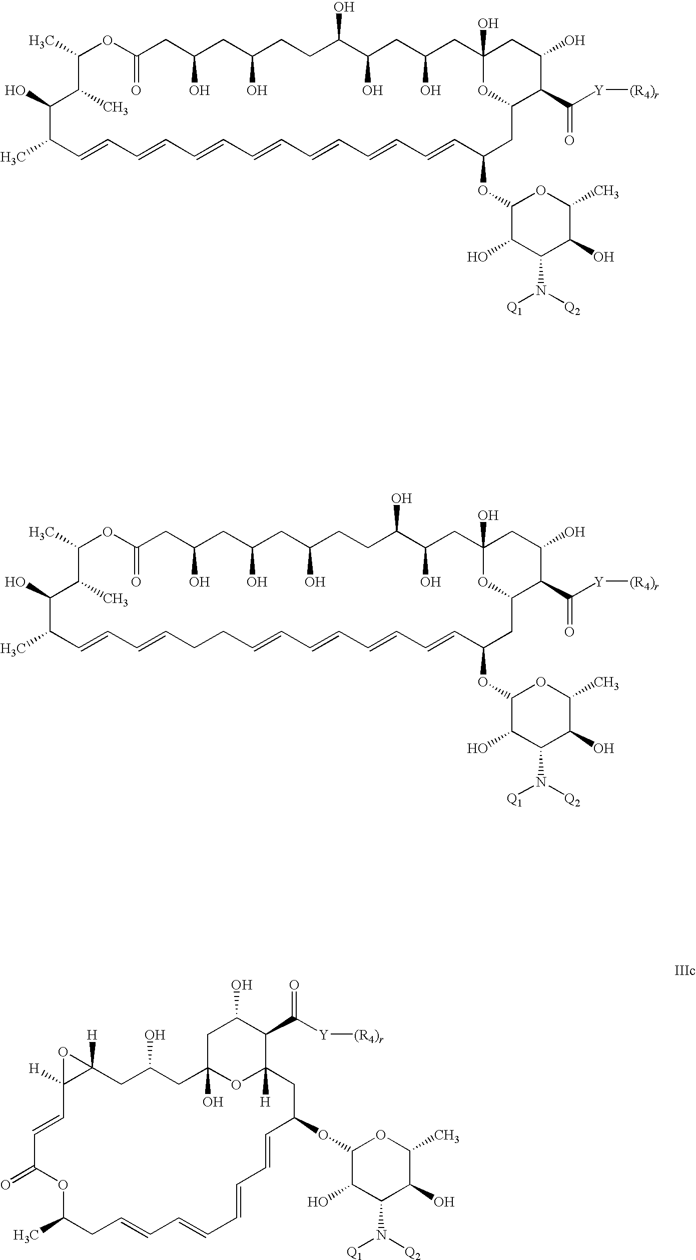
wherein:
Q₁ and Q₂ represent
(i) a group of formula —(R₁)—(X₁)ₚ and —(R₂)—(X₂)_{q}, respectively, wherein
X₁, X₂ represent independently of each other a basic group, preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
R₁, R₂ represent independently of each other an unsubstituted or substituted hydrocarbon group, selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; or
(ii) taken together with the adjacent nitrogen atom to which they are attached, a nitrogen-containing heterocyclic group substituted with at least one substituent of formula —(R₃)—(X₃)ₒ, wherein R₃ and X₃ have the same meaning as R₁ and X₁, respectively, and o has the meaning of m+n and represents 2, 3 or 4;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, CH═CH— or —C≡C—, wherein R₅ independently represents hydrogen or alkyl; and
r is 1 or 2.

10. Polyene macrolide according to claim 1 having the structure of formula IV: 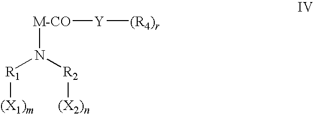
or a pharmaceutically acceptable salt thereof,
wherein:
M represents a polyene macrolide backbone;
R₁, R₂ represent independently of each other linear or branched —(CH₂)ₚ—, wherein p is an integer from 0 to 12 and in which one or more —CH₂— groups are optionally replaced by —O—, —CO—, —COO—, —CONR₅—, —NR₅—, —CH═CH—, wherein R₅ independently represents hydrogen or alkyl;
X₁, X₂ represent independently of each other a basic group, which may be attached to any —CH₂— group of R₁ and R₂, respectively, and is preferably selected from —N(R₅)₂, —OH, —SH, —C(═NR₅)—N(R₅)₂, —NR₅—C(═NR₅)—N(R₅)₂, —N₃, —COR₅, —CSR₅, —COOR₅, —CONHR₅, and —CN, wherein R₅ represents hydrogen or alkyl;
m, n represent independently of each other 0, 1 or 2, with m+n≧2,
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

11. Polyene macrolide derivatives according to claim 10 having the structure of formula V: 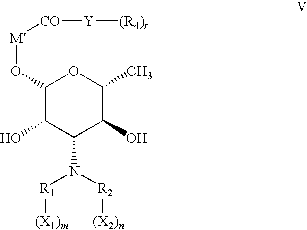
or a pharmaceutically acceptable salt thereof,
wherein:
M′ represents the macrocyclic lactone ring of a polyene macrolide backbone;
and R₁, R₂, R₄, X₁ and X₂, Y, m, n, and r are as defined in claim 10.

12. Polyene macrolide derivatives according to claim 10 having the structure of formulae VIa-e, 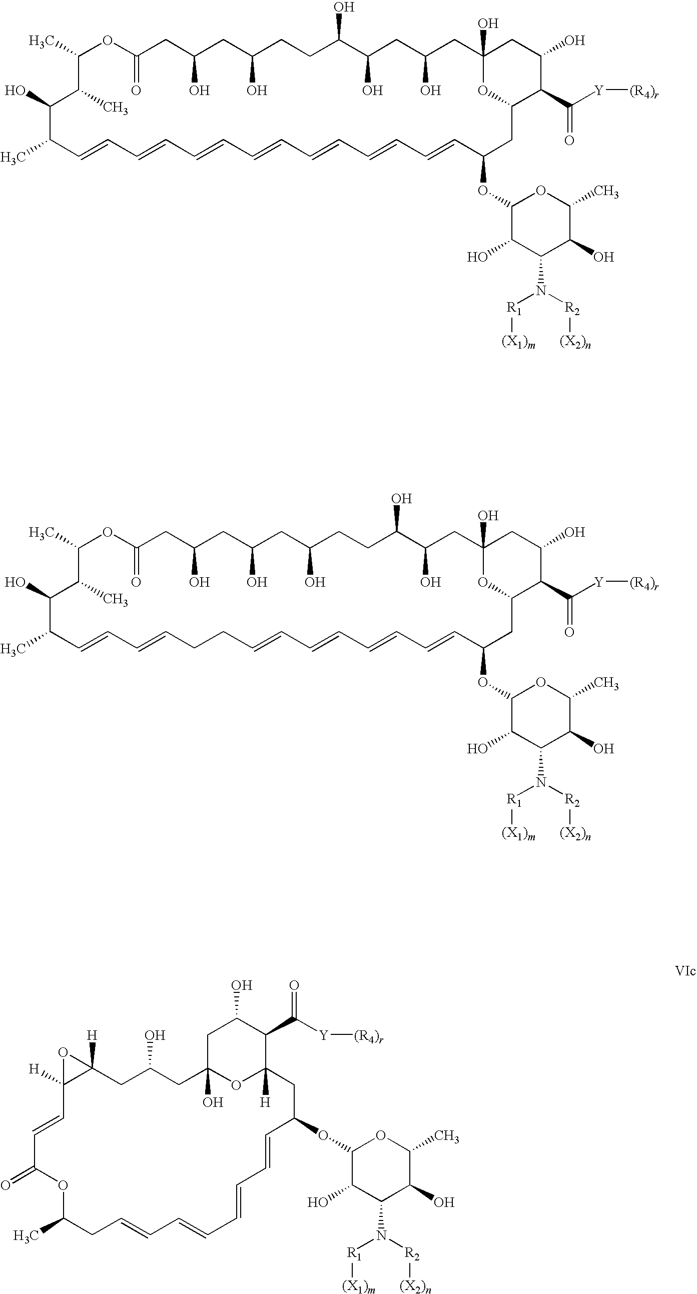
or a pharmaceutically acceptable salt thereof, wherein:
R₁, R₂, R₄, X₁ and X₂, Y, m, n, and r are as defined in claim 10.

13. Polyene macrolide according to claim 1 having the structure of formula VII: 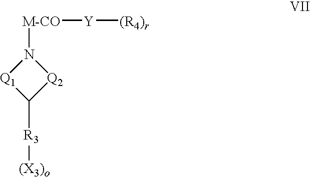
or a pharmaceutically acceptable salt thereof,
wherein:
M represents a polyene macrolide backbone;
Q₁, Q₂ form together with the adjacent nitrogen atom to which they are attached a nitrogen-containing heterocyclic group;
X₃ represents a basic group, which may be attached to any —CH₂— group of R₃, preferably selected from —N(R₅)₂, —OH, —SH, —C(═NR₅)—N(R₅)₂, —NR₅—C(═NR₅)—N(R₅)₂, —N₃, —COR₅, CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

14. Polyene macrolide derivatives according to claim 13 having the structure of formula VIII: 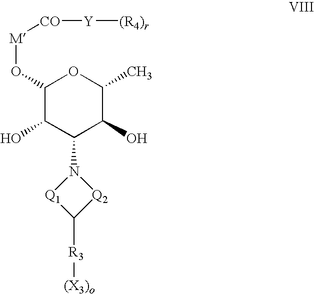
or a pharmaceutically acceptable salt thereof,
wherein:
M′ represents the macrocyclic lactone ring of a polyene macrolide backbone;
and Q₁, Q₂, R₃, R₄, X₃, Y, o, and r are as defined in claim 13.

15. Polyene macrolide derivatives according to claim having the structure of formulae IX a-c, 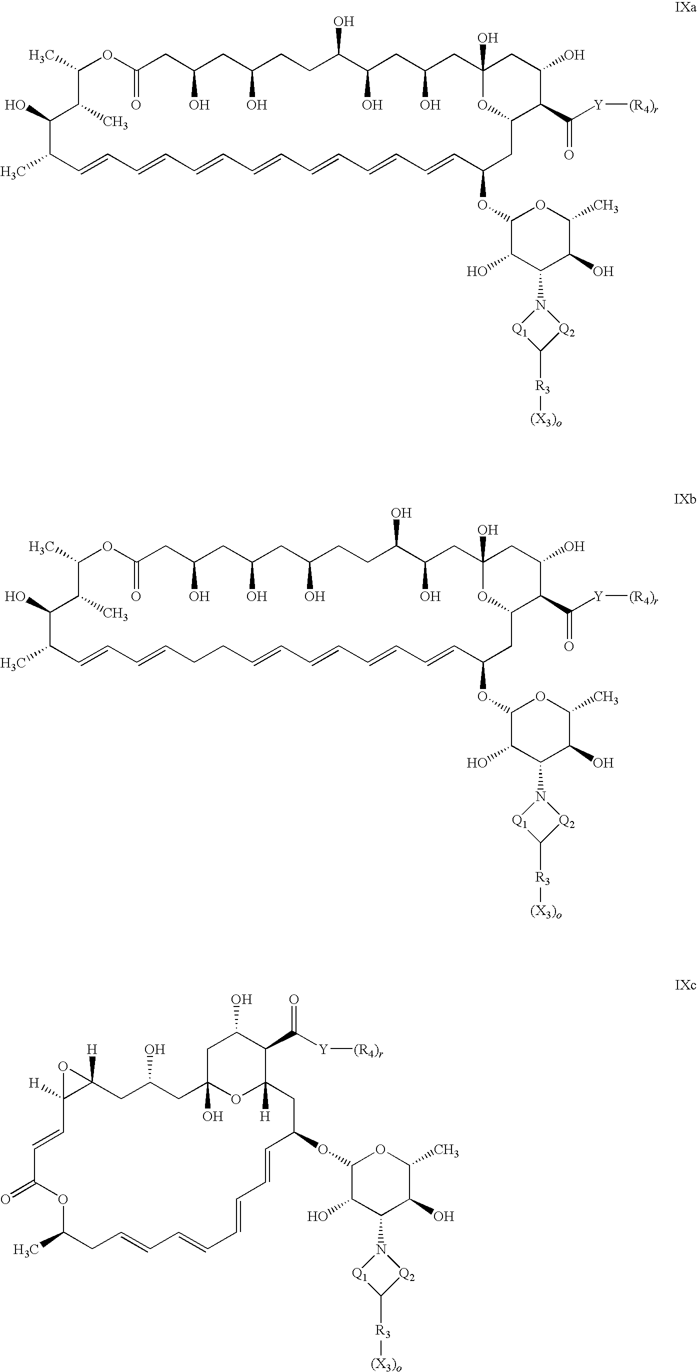
or a pharmaceutically acceptable salt thereof, wherein:
Q₁, Q₂, R₃, R₄, X₃, Y, o, and r are as defined in claim 13.

16. Polyene macrolide according to claim 1 having the structure of formula X: 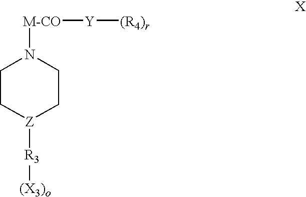
or a pharmaceutically acceptable salt thereof,
wherein:
M represents a polyene macrolide backbone;
Z represents —CH— or —N—;
X₃ represents a basic group, which may be attached to any —CH₂— group of R₃, preferably selected from —N(R₅)₂, —OH, —SH, —C(═NR₅)—N(R₅)₂, —NR₅—C(═NR₅)—N(R₅)₂, —N₃, —COR₅, CSR₅, —COOR₅, —CONHR₅, and —CN, wherein R₅ represents hydrogen or alkyl;
o represents at least 2, preferably 2, 3 or 4,
R₃ represents an unsubstituted or substituted hydrocarbon group, attached to any site of the heterocyclic group formed by Q₁, Q₂ and N, selected from alkyl, cycloalkyl, heterocycloalkyl, aryl, heteroaryl, arylalkyl and heteroarylalkyl groups, in which one or more —CH₂-groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl;
Y represents O, S, N or NH,
R₄ represents hydrogen or an unsubstituted or substituted hydrocarbon group, preferably selected from alkyl, cycloalkyl, heterocycloalkyl, aryl, heteroaryl, arylalkyl and heteroarylalkyl groups, in which one or more —CH₂— groups of the alkyl groups are optionally replaced by a group selected from —O—, —CO—, —COO—, —OCO—, —O—CO—O—, —NR₅—, —NR₅CO—, —NR₅—COO—, —C(═NH)—NH—, —CH═CH— or —C≡C—, wherein R₅ independently represents hydrogen or alkyl; and
r is 1 or 2.

17. Polyene macrolide according to claim 16 having the structure of formula XI: 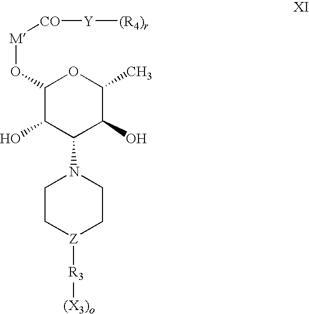
or a pharmaceutically acceptable salt thereof,
wherein:
M′ represents the macrocyclic lactone ring of a polyene macrolide backbone;
and R₃, R₄, X₃, Z, Y, o and r are as defined in claim 16.

18. Polyene macrolide according to claim 16 having the structure of formulae XII a-c, 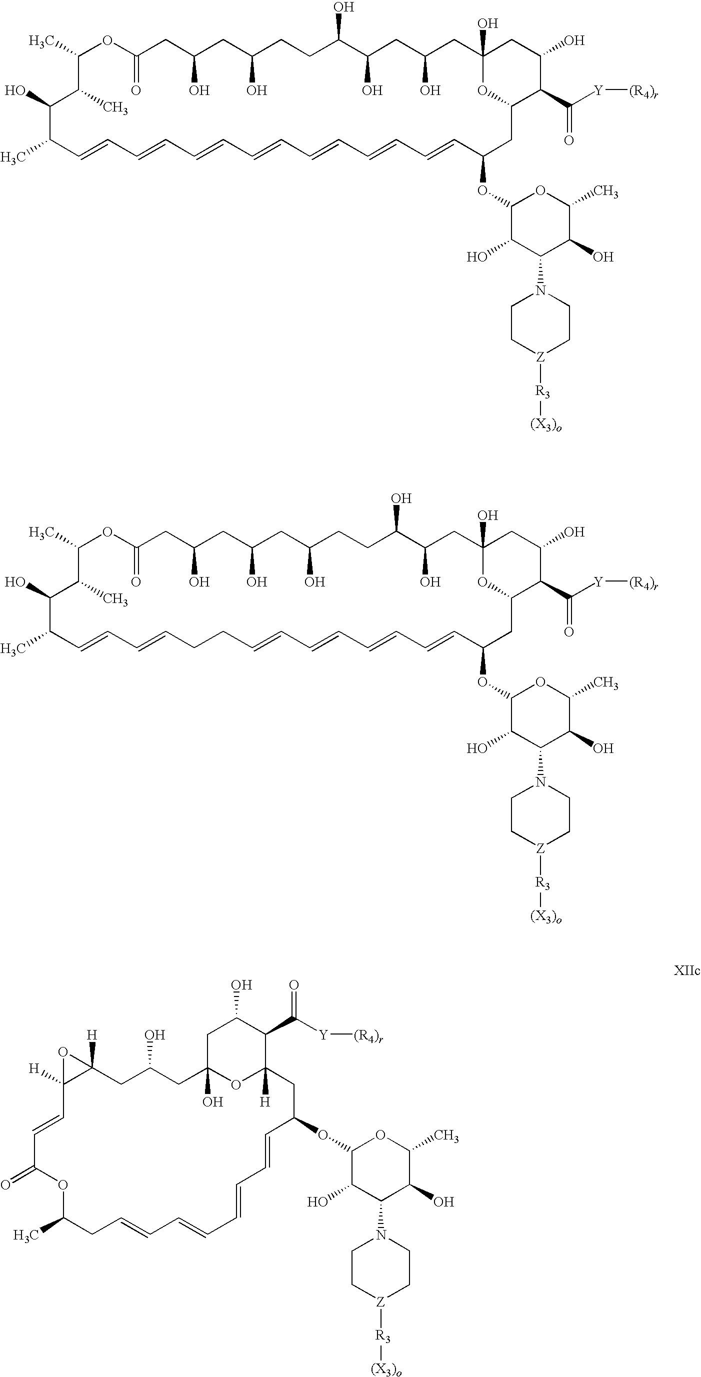
or a pharmaceutically acceptable salt thereof, wherein:
R₃, R₄, X₃, Z, Y, o and r are as defined in claim 16.

19. Method of producing a polyene macrolide derivative according to claims 1 to 12, comprising subjecting a polyene macrolide to double reductive alkylation with two optionally protected functionalized aldehydes of formula P—(X₁)ₘ—(R₁)—CHO and P— (X₂)ₙ—(R₂)—CHO wherein X₁, X₂, R₁ and R₂, m and n are as defined hereinabove and P is H or a suitable protecting group.

20. Method of producing a polyene macrolide derivative according to claims 13 to 18, comprising subjecting a polyene macrolide to double reductive alkylation with an optionally protected functionalized aldehyde P—(X₃)ₒ—(R₃)—(CHO)₂, wherein X₃, R₃, and o are as defined hereinabove and P is H or a suitable protecting group.

21. Pharmaceutical composition comprising at least one polyene macrolide derivative according to claims 1 to 18 and a pharmaceutically acceptable carrier.

22. A unit dosage form comprising polyene macrolide derivative according to claims 1 to 18 or one or more pharmaceutical compositions according to claim 21 for pharmaceutical use.

23. A pharmaceutical composition according to claim 21 or a unit dosage form according to claim 22 which further comprises at least one further pharmaceutically active agent.

24. A pharmaceutical composition according to claim 21 or a unit dosage form according to claim 22 which is formulated for intravenous, intraperitoneal, oral, topical, subcutaneous, rectal or vaginal administration.

25. Method of inhibiting the growth of fungi, which methods comprise contacting a fungus with an effective amount of a polyene macrolide derivative according to claims 1 to 18, or a pharmaceutically acceptable salt thereof, or a pharmaceutical composition according to claim 21 to inhibit the growth of the fungus.

26. Polyene macrolide derivative according to claims 1 to 18 or pharmaceutical compositions according to claim 21 for use in therapy.

27. Method for the treatment and/or prevention of a fungal infection in a subject, comprising administering to a subject in need of such treatment and/or prevention at least one polyene macrolide derivative according to claims 1 to 18 or pharmaceutical compositions according to claim 21, in therapeutically effective amounts.

28. (canceled)

29. (canceled)

30. Kit for use in exercising the methods of the present invention comprising at least one polyene macrolide derivative according to claims 1 to 18 or pharmaceutical composition according to claim 21 and optional other pharmaceutically active agents or pharmaceutical formulations thereof in one or more vials.
